(19) United States
(12) Patent Application Publication
(10) Pub. No.: US 2003/0224989 A1
(43) Pub. Date: Dec. 4, 2003

(54) Compositions and methods for treatment of osteoarthritis

(76) Inventors: Kerry Quinn, Hamden, CT (US); Gregory L. Pahel, Morrisville, NC (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/364,889

(22) Filed: Feb. 12, 2003

Related U.S. Application Data
(60) Provisional application No. 60/356,376, filed on Feb. 12, 2002.

(51) Int. Cl. A61K 38/17

(52) U.S. Cl. 514/12

(57) ABSTRACT

The present invention is based upon methods of treating musculoskeletal or pro-inflammatory conditions and pathologies in mammals using. Methods of using the polynucleotide sequences and the the polypeptides encoded by such nucleic acid sequences, or variants, fragments and homologs thereof, are claimed in the invention. Similarly, methods of using OAX polynucleotide sequences and the OAX polypeptides encoded by such nucleic acid sequences, or variants, fragments and homologs thereof, alone or in combination, are also claimed in the invention. OAX collectively refers to any of six variant OAX sequences, variously designated OA1, OA2, OA3, and OA4.

### RELATED APPLICATION

This application claims the benefit of priority from U.S. Ser. No. 60/356,376 filed Feb. 12, 2002, the contents of which are incorporated herein in its entirety.

### FIELD OF THE INVENTION

The present invention generally relates to methods of treatment of musculoskeletal conditions in mammals using polypeptides and nucleic acid sequences encoding them. More specifically, the polypeptides employed in the methods of the invention are differentially expressed proteins in osteoarthritic samples.

### BACKGROUND OF THE INVENTION

Osteoarthritis (OA) is the most prevalent form of arthritis. In the United States, it affects 20 million people and is predicted to affect 40 million people by the year 2020, as the population ages. The medical costs attributed to OA represent 15 billion dollars annually. The substantial prevalence of OA in the middle-aged population also causes considerable burden in lost working time and early retirement. Musculoskeletal conditions such as OA cost the U.S. economy nearly $65 billion per year in direct expenses and lost wages and productivity.

The pathology of osteoarthritis involves the whole joint. Characteristic features of OA include focal and progressive hyaline articular cartilage loss with concomitant changes in the bone underneath the cartilage, including development of marginal outgrowths, osteophytes and increase in subchondral bone thickness. Soft tissues in and around the cartilage are also affected, including the synovium, which may show modest inflammatory infiltrates, and the ligaments, which are often lax. The exact etiology of OA is not known but there are several possible causes including: injury, age, congenital predisposition and obesity.

In osteoarthritis, the fundamental event resulting in progressive loss of articular cartilage arises from an imbalance between the anabolic and catabolic pathways. The extracellular matrix of cartilage is degraded by matrix metalloproteinases (MMPs) that are induced by cytokines. In osteoarthritic cartilage, matrix degrading enzymes are overexpressed, shifting this balance in favor of net degradation, with resultant loss of collagen and proteoglycans from the matrix. Presumably in response to this loss, chondrocytes initially proliferate and synthesize enhanced amounts of proteoglycan and collagen molecules. As the disease progresses, however, reparative attempts are outmatched by progressive cartilage degradation. Fibrillation, erosion and cracking initially appear in the superficial layer of cartilage and progress over time to deeper layers, resulting eventually in large clinically observable erosions. OA, in simplistic terms, therefore, can be thought of as a process of progressive cartilage matrix degradation.

Growth factors are produced locally in cartilage and synovium and are likely to contribute to local cartilage remodeling by stimulating the de novo synthesis of collagen and proteoglycans. Synthesis and secretion of growth factors and of inhibitors of MMPs and cytokines are apparently inadequate to counteract these degradative forces. Progressive cartilage degradation occurs and OA results. New therapies focused on reducing MMP activity and on stimulating matrix synthesis.

Current treatments for osteoarthritis focus on decreasing pain and improving joint movement, and include:

(1) Exercises to keep joints flexible and improve muscle strength

(2) Medications used to control pain, including corticosteroids and NSAIDs. Glucocorticoids are injected into joints that are inflamed and not responsive to NSAIDS. For mild pain without inflammation, acetaminophen may be used.

(3) Cyclooxygenase II inhibitors

(4) Heat/cold therapy for temporary pain relief

(5) Joint protection to prevent strain or stress on painful joints

(6) Surgery may occasionally be performed to relieve chronic pain in damaged joints

(7) Weight control to prevent extra stress on weight-bearing joints

Currently not one single test can pinpoint the disease. A combination of patient history, exam, and x-rays are commonly used by physicians to diagnose the disease and rule out other causes for the symptoms. Thus, no effective therapies currently exist that modulate the key markers of OA disease progression. The two mains areas of focus for development of OA therapies are to either prevent or slow down extracellular matrix degradation (by generating MMP inhibitors or inhibitors of MMP inducers) or to promote cartilage repair.

Consequently a therapeutic that can successfully diagnose and treat inflammatory or pro-inflammatory conditions will have the beneficial effects of improving a patient's quality of life, while potentially saving the healthcare system millions of dollars in costs associated with invasive surgical procedures.

### SUMMARY OF THE INVENTION

The present invention is directed to methods of diagnosing and treating inflammatory conditions or degeneration in the cartilage and tissues of mammals using proteins or mixtures of proteins as therapeutic compositions. More specifically, the invention is directed to methods of diagnosing and treating osteoarthritis. Methods of using OAX polynucleotide sequences and the OAX polypeptides encoded by such nucleic acid sequences, or variants, fragments and homologs thereof, alone or in combination, are claimed in the invention. OAX collectively refers to any of eight variant OAX sequences, designated OA1, OA2, OA3 and OA4.

In one aspect, the invention provides a method of promoting the growth of a population of cells whereby the cells are placed into contact with a composition including an OAX polypeptide. In another aspect, the invention provides a method of treating a musculoskeletal condition in a subject, whereby an OAX polypeptide composition is administered to the subject. In yet another aspect, the invention provides a method of delaying the onset of an inflammatory pathology in a subject, whereby a composition including an OAX polypeptide is administered to the subject. In a further aspect, the invention provides a method of diagnosing a predisposition to a pro-inflammatory or inflammatory pathology in a subject, whereby a composition including an OAX polypeptide, or a composition including OAX polypeptides, is detected in a subject.

In one embodiment, the subject is a mammal. In another embodiment, the subject is human. In yet another embodiment, the inflammatory pathology includes osteoarthritis, inflammation of the joint and joint space, and degeneration of the cartilage matrix. In yet another embodiment, the OAX polypeptide is given by the sequences described below or a variant, deletion mutant, or a variant of the deletion mutant thereof, wherein up to 15% of the residues of either variant are changed according to a conservative amino acid substitution. In still yet another embodiment, the OAX polypeptide is given by any one of SEQ ID NOS:2, 4, 6, 8 or a variant, deletion mutant, variant of the deletion mutant, wherein up to 15% of the residues of any variant are changed according to a conservative amino acid substitution. In yet a further embodiment, the polypeptide composition is administered intravenously or subcutaneously.

The invention further provides a method of preparing a pharmaceutical composition, hereby a polypeptide effective in treating a musculoskeletal pathology is combined with a pharmaceutically acceptable carrier.

In one embodiment, the pharmaceutical composition is suitable for intravenous, or subcutaneous administration to the subject. In another embodiment, the polypeptide is OAX. In yet another embodiment, the OAX polypeptide is given by SEQ ID NO:2, 4, 6, 8, or a variant, deletion mutant, or a variant of the deletion mutant thereof, wherein up to 15% of the residues of either variant are changed according to a conservative amino acid substitution. In a further embodiment, the polypeptide is OAX. In yet a further embodiment, the OAX polypeptide is given by any one of SEQ ID NOS: 2, 4, 6, 8 or a variant, deletion mutant, variant of the deletion mutant, wherein up to 15% of the residues of any variant are changed according to a conservative amino acid substitution

Contemplated disorders within the invention include pathologies such as inflammatory joint disease, joint disease, or degenerative arthritis. In addition, OAX nucleic acids and their encoded polypeptides will be therapeutically useful for the prevention of inflammation and degeneration in tissues and cartilage through gene therapy. Furthermore, OAX nucleic acids and their encoded polypeptides can be utilized to treat inflammatory conditions associated with osteoarthritis, inflammation of the joint and joint space, and degeneration of the cartilage matrix, to promote repair of the cartilage matrix (i.e., to promote the synthesis and deposition of oligomatrix support proteins) and to promote chondrocyte growth and development and reduce cellular apoptosis of chondrocyte.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, issued patents, and other references mentioned herein are incorporated by reference in their entirety. In the case of conflict, the present specification, including definitions, will control. In addition, the materials, methods, and examples are illustrative only and not intended to be limiting. Other features and advantages of the invention will be apparent from the following detailed description and claims.

Other features and advantages of the invention will be apparent from the following detailed description and claims.

### BRIEF DESCRIPTION OF THE DRAWINGS

FIG. 1 shows Lysyl Oxidase quantitative expression analysis (QEA) trace data.

FIG. 2 shows A) the gene sequence for Stanniocalcin-2 and B) Stanniocalcin-2 quantitative expression analysis (QEA) trace data.

FIG. 3 shows A) the gene sequence for Adrenomedullin and B) Adrenomedullin quantitative expression analysis (QEA) trace data.

FIG. 4 shows A) the gene sequence for Latent Transforming Growth Factor-beta-binding protein-2 (LTBP2) and B) LTBP2 quantitative expression analysis (QEA) trace data

### DETAILED DESCRIPTION OF THE INVENTION

This invention is related in part to the discovery of the novel association of OAX nucleic acid sequences and polypeptides with musculoskeletal conditions. An OAX nucleic acid or gene product, is useful as a therapeutic agent in promoting cartilage repair, reducing inflammation, promoting chondrocyte growth and development and reduce cellular apoptosis of chondrocyte. OAX play a role in diagnosing or treating osteoarthritis. As used herein the designation “OAX” relates to nucleic acids, polynucleotides, proteins, polypeptides, and variants, derivatives and fragments of any of them, as well as to antibodies that bind immunospecifically to any of these classes of compounds.

The invention further is based on the discovery of nucleic acids that encode polypeptides differentially expressed in osteoarthritic samples designated as “OAX nucleic acids” or OAX polynucleotides” and the corresponding encoded polypeptide is referred to as a “OAX polypeptide” or “OAX protein.” Unless indicated otherwise, “OAX” is meant to refer to any of the sequences disclosed herein.

The OAX nucleic acid and polypeptides described below as OA1-OA4 can be used with the methods of the present invention to ameliorate or treat the pathologies associated with osteoarthritis. By “treating” is meant the administration of a protein used in the present invention to a subject suffering from a pathology such as osteoarthritis with the objective of providing a beneficial therapeutic effect. By “ameliorating” a pathology such as osteoarthritis, it is meant that a) in a subject in which the pathology is becoming more severe, one or more symptoms of the pathology cease becoming more severe and stabilize or improve; or b) in a subject in which the pathology is considered to be at a stable state, one or more symptoms of the pathology improve or become less severe. By “delaying the onset” of a pathology such as osteoarthritis, it is meant that administering a prophylactic dose or dosing regimen of a therapeutic agent such as the OAX proteins employed in the present invention results in the delay of appearance, or the delay of worsening, of one or more symptoms of a pathology such as osteoarthritis. Such a delay may be for an indeterminate period, in which the symptoms essentially never appear or never worsen, or it may be for a more limited period, in which the symptoms appear or worsen at a later time than would be expected, based on the experience of patients not treated by the compositions envisioned in the present methods, in the absence of administering the therapeutic agent.

OA1

A polynucleotide to be used with the methods of the invention includes the nucleic acid of OA1 (also referred to as Human Lysyl Oxidase GenBabnk Accession No. AF039291). OA1 is 1935 nucleotides in length (for the total sequence shown) and 1254 nucleotides in length (for the translated region). The nucleotide sequence of OA1 is reported in Table 1 (SEQ ID NO:1). The untranslated regions upstream of the start site and downstream of the stop codon are underlined, and the start and stop codons are shown in bold.

TABLE 1

Nucleotide (SEQ ID NO: 1 and Protein (SEQ ID NO: 2)
Sequence of OA1

| | |
|---|---|
| Frame: +1 - Nucleotide 250 to 1500 | |
| CCGCGCCGCTCCCCGTTGCCTTCCAGGACTGAGAAAGGGGAAAGGGAAGGGTGCCACGTCCG | (Seq Id No. 1) |
| AGCAGCCGCCTTGACTGGGGAAGGGTCTGAATCCCACCCTTGGCATTGCCTGGTGGAGACTG | |
| AGATACCCGTGCTCCGCTCGCCTCCTTGGTTGAAGATTTCTCCTTCCCTCACGTGATTTGAG | |
| CCCCGTTTTTATTTTCTGTGAGCCACGTCCTCCTCGAGCGGGGTCAATCTGGCAAAAGGAGT | |
| GATGCGCTTCGCCTGGACCGTGCTCCTGCTCGGGCCTTTGCAGCTCTGCGCGCTAGTGCACT | |
| GCGCCCCTCCCGCCGCCGGCCAACAGCAGCCCCCGCGCGAGCCGCCGGCGGCTCCGGGCGCC | |
| TGGCGCCAGCAGATCCAATGGGAGAACAACGGGCAGGTGTTCAGCTTGCTGAGCCTGGGCTC | |
| ACAGTACCAGCCTCAGCGCCGCCGGGACCCGGGCGCCGCCGTCCCTGGTGCAGCCAACGCCT | |
| CCGCCCAGCAGCCCCGCACTCCGATCCTGCTGATCCGCGACAACCGCACCGCCGCGGCGCGA | |
| ACGCGGACGGCCGGCTCATCTGGAGTCACCGCTGGCCGCCCCAGGCCCACCGCCCGTCACTG | |
| GTTCCAAGCTGGCTACTCGACATCTAGAGCCCGCGAAGCTGGCGCCTCGCGCGCGGAGAACC | |
| AGACAGCGCCGGGAGAAGTTCCTGCGCTCAGTAACCTGCGGCCGCCCAGCCGCGTGGACGGC | |
| ATGGTGGGCGACGACCCTTACAACCCCTACAAGTACTCTGACGACAACCCTTATTACAACTA | |
| CTACGATACTTATGAAAGGCCCAGACCTGGGGGCAGGTACCGGCCCGGATACGGCACTGGCT | |
| ACTTCCAGTACGGTCTCCCAGACCTGGTGGCCGACCCCTACTACATCCAGGCGTCCACGTAC | |
| GTGCAGAAGATGTCCATGTACAACCTGAGATGCGCGGCGGAGGAAAACTGTCTGGCCAGTAC | |
| AGCATACAGGGCACATGTCAGAGATTATGATCACAGGGTGCTGCTCAGATTTCCCCAAAGAG | |
| TGAAAAACCAAGGGACATCAGATTTCTTACCCAGCCGACCAAGATATTCCTGGGAATGGCAC | |
| AGTTGTCATCAACATTACCACAGTATGGATGAGTTTAGCCACTATGACCTGCTTGATGCCAA | |
| CACCCAGAGGAGAGTGGCTGAAGGCCACAAAGCAAGTTTCTGTCTTGAAGACACATCCTGTG | |
| ACTATGGCTACCACAGGCGATTTGCATGTACTGCACACACACAGGGATTCAGTCCTGGCTGT | |
| TATGATACCTATGGTGCAGACATAGACTGCCAGTGGATTGATATTACAGATGTAAAACCTGG | |
| AAACTATATCCTAAAGGTCAGTGTAAACCCCAGCTACCTGGTTCCTGAATCTGACTATACCA | |
| ACAATGTTGTGCGCTGTGACATTCGCTACACAGGACATCATGCGTATGCCTCAGGCTGCACA | |
| ATTTCACCGTATTAGAAGGCAAAGCAAAACTCCCAATGGATAAATCAGTGCCTGGTGTTCTG | |
| AAGTGGGAAAAAATAGACTAACTTCAGTAGGATTTATGTATTTTGAAAAAGAGAACAGAAAA | |
| CAACAAAAGAATTTTTGTTTGGACTGTTTTCAATAACAAAGCACATAACTCGATTTTGAACG | |
| CTTAAGTCATCATTACTTGGGAAATTTTTAATGTTTATTATTTACATCACTTTGTGAATTAA | |
| CACAGTGTTTCAATTCTGTAATTACATATTTGACTCTTTCAAAGAAATCCAAATTTCTCATG | |
| TTCCTTTTGAAATTGTAGTGCAAAATGGTCAGTATTATCTAAATGAATGAGCCAAAATGACT | |
| TTGAACTGAAACTTTTCTAAAGTGCTGGAACTTTAGTGAAACATAATAATAATGGGTTTATA | |
| CGACAGCAACGGA | |
| Protein Translation: 417 amino acid reading frame | |
| MRFAWTVLLLGPLQLCALVHCAPPAAGQQQPPREPPAAPGAWRQQIQWENNGQVFSLLSLGS | (Seq Id. 2) |
| QYQPQRRRDPGAAVPGAANASAQQPRTPILLIRDNRTAAARTRTAGSSGVTAGRPRPTARHW | |
| FQAGYSTSRAREAGASRAENQTAPGEVPALSNLRPPSRVDGMVGDDPYNPYKYSDDNPYYNY | |
| YDTYERPRPGGRYRPGYGTGYFQYGLPDLVADPYYIQASTYVQKMSMYNLRCAAEENCLAST | |
| AYRADVRDYDHRVLLRFPQRVKNQGTSDFLPSRPRYSWEWHSCHQHYHSMDEFSHYDLLDAN | |
| TQRRVAEGHKASFCLEDTSCDYGYHRRFACTAHTQGLSPGCYDTYGADIDCQWIDITDVKPG | |
| NYILKVSVNPSYLVPESDYTNNVVRCDIRYTGHHAYASGCTISPY | |

Nucleotides 250-1500 of SEQ ID NO:1 encodes a 417 amino acid protein (SEQ ID NO:2) that includes sequences characteristic of secreted proteins. The sequence of the encoded protein, which is also referred to herein as “OA 1 protein,” is presented in Table 1.

The human lysyl oxidase gene, OA1, has been mapped to 5q23.3-q31.2. Disorders mapped to this locus that are not already linked to a gene have been identified in Cutis Laxa, Recessive, Type I (which is a deficiency of elastic fibers affecting several tissues. Reported cases include diverticula in the GI tract and pulmonary emphysema. OMIM: 219100).

BLASTN and BLASTP analyses indicate the OA1 polypeptide has an entry for a BLASTN match to GENBANK-ID:S78694, Collagen Research Unit, University of Oulu, Finland.

Lysyl oxidase, OA1, is an extracellular copper enzyme, which initiates the crosslinking of collagens and elastin by catalyzing oxidative deamination of the epsilon-amino group in certain lysine and hydroxylysine residues. A deficiency in lysyl oxidase activity has been found in 3×-linked recessive human connective tissue disorders known as cutis laxa (formerly Ehlers-Danlos syndrome type IX, Menkes syndrome, and Ehlers-Danlos syndrome type V). The deficiency is also associated with Primary cutis laxa and osteoporosis. (OMIM: 153455).

The present invention has identified down-regulation of the expression of lysyl oxidase, OA1, in human dermal fibroblasts cultured under conditions of high mechanical stress. Since lysyl oxidase is involved in extracellular catalysis of lysine-derived cross-links in fibrillar collagens and elastin, down-regulation in response to mechanical tension may result in modulating the stiffness of the extracellular matrix [PMID: 11499790]. Treatment with OA1 applied as a protein therapeutic may help to slow the progression of OA by restoring an extracellular matrix with improved biomechanical properties. Further, by mediating the cross-linking of collagen and elastin, OA1 may participate in the rebuilding of osteoarthritis-damaged cartilage matrix in joints. Accordingly, an inflammatory pathology in a subject can be treated by administering to the subject a composition copmprising an OA1 polypeptide. The inflammatory pathology to be treated includes osteoarthritis, and inflammation of the joint and joint space, and degeneration of the cartilage matrix. promoting repair of the cartilage matrix (i.e., to promote the synthesis and deposition of oligomatrix support proteins) and for promoting chondrocyte growth and development and reduce cellular apoptosis of chondrocyte. Additional disorders wherein the associated expression of OAX in inflammatory conditions include those that mediate pro-inflammatory events such as inflammatory bowel disease (Crohns disease and ulcerative colitis), inflammatory lung disorders (asthma, chronic obstructive pulmonary disease (COPD), cystic fibrosis (CF), bronchiectasis and interstitial lung diseases) as well as Sjogren's syndrome, psoriasis, and rheumatoid arthritis.

A fragment of the human Lysyl Oxidase gene (GenBank ID #AF039291 and confirmed via a fragment identified for SeqCalling ID sch_gb-116895_{—}1) was found to be up-regulated in normal cartilage relative to cartilage from osteoarthritic knees using CuraGen's GeneCalling™ method of differential gene expression (see Example 1, infra). Sch_gb-116895_{—}1 was confirmed as identical to the 3′ end of the full length sequence for the human lysyl oxidase gene (gbh_AG039291).

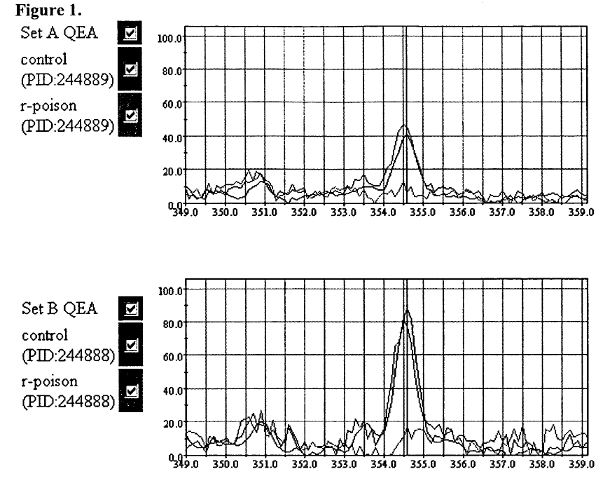

A differentially expressed human gene fragment migrating at approximately 354.6 nucleotides (nt) in length (FIG. 1.—solid vertical line) was definitively identified as a component of the human Lysyl Oxidase cDNA (in the graphs, the abscissa is measured in lengths of nt and the ordinate is measured as signal response. Refer to traces in FIG. 1 (“QEA” serves as the original trace and “control” shows the recapitulation of the QEA following an independent chemistry reaction. Peak height differences between set A and B is used to calculate the N-fold difference is expression. The method of comparative PCR was used for confirmation of the gene assessment. The electropherographic peaks corresponding to the gene fragment of the human Lysyl Oxidase are ablated when a gene-specific primer competes with primers in the linker-adaptors during the PCR amplification. The peaks at 354.6 nt in length are ablated in the samples of human cartilage from normal (Set B) and osteoarthric (Set A) knees (refer to “r-poison” trace in FIG. 1). Detectable expression of this gene was also detected by RTQ-PCR of samples from a number of tissues from osteoarthritic patients.

Taken together, these data show that Lysyl Oxidase is down-regulated in osteoarthritic cartilage. Thus, treatment with lysyl oxidase applied as a protein therapeutic can be utilized in the treatment of osteoarthritis and may help slow the progression of OA.

The OAX nucleic acids and polypeptides, as well as OAX antibodies, therapeutic agents and pharmaceutical compositions discussed herein, are useful, inter alia, in treating inflammatory conditions associated with osteoarthritis, inflammation of the joint and joint space, degeneration of the cartilage matrix, promoting repair of the cartilage matrix (i.e., to promote the synthesis and deposition of oligomatrix support proteins) and for promoting chondrocyte growth and development and reduce cellular apoptosis of chondrocyte.

OA2 Nucleic Acids and Polypeptides

A polynucleotide to be used with methods of the present invention includes the nucleic acid sequence of OA2 (also referred to as Human Stanniocalcin-2 (STC2), sch_gb-af098462_{—}1, or GenBank Accession No. AF055460). OA2 is 1837 nucleotides in length (for the total sequence shown) and 909 nucleotides in length (for the translated region). The nucleotide sequence of OA2 is shown in Table 2 (SEQ ID NO:3). The untranslated regions upstream of the start site and downstream of the stop codon are underlined, and the start and stop codons are shown in bold.

TABLE 2

Nucleotide (SEQ ID NO: 3) and Protein (SEQ ID NO: 4)
Sequence of OA2

| | |
|---|---|
| Nucleotide 123 to 1028 | |
| CGGCACGAGCAAAAAGGAAGAGTGGGAGGAGGAGGGGAAGCGGCGAAGGAGGAAGAGGA | (Seq Id No. 3) |
| GGAGGAGGAAGAGGGGAGCACAAAGGATCCAGGTCTCCCGACGGGAGGTTAATACCAAG | |
| AACCATGTGTGCCGAGCGGCTGGGCCAGTTCATGACCCTGGCTTTGGTGTTGGCCACCT | |
| TTGACCCGGCGCGGGGGACCGACGCCACCAACCCACCCGAGGGTCCCCAAGACAGGAGC | |
| TCCCAGCAGAAAGGCCGCCTGTCCCTGCAGAATACAGCGGAGATCCAGCACTGTTTGGT | |
| CAACGCTGGCGATGTGGGGTGTGGCGTGTTTGAATGTTTCGAGAACAACTCTTGTGAGA | |
| TTCGGGGCTTACATGGGATTTGCATGACTTTTCTGCACAACGCTGGAAAATTTGATGCC | |
| CAGGGCAAGTCATTCATCAAAGACGCCTTGAAATGTAAGGCCCACGCTCTGCGGCACAG | |
| GTTCGGCTGCATAAGCCGGAAGTGCCCGGCCATCAGGGAAATGGTGTCCCAGTTGCAGC | |
| GGGAATGCTACCTCAAGCACGACCTGTGCGCGGCTGCCCAGGAGAACACCCGGGTGATA | |
| GTGGAGATGATCCATTTCAAGGACTTGCTGCTGCACGAACCCTACGTGGACCTCGTGAA | |
| CTTGCTGCTGACCTGTGGGGAGGAGGTGAAGGAGGCCATCACCCACAGCGTGCAGGTTC | |
| AGTGTGAGCAGAACTGGGGAAGCCTGTGCTCCATCTTGAGCTTCTGCACCTCGGCCATC | |
| CAGAAGCCTCCCACGGCGCCCCCCGAGCGCCAGCCCCAGGTGGACAGAACCAAGCTCTC | |
| CAGGGCCCACCACGGGGAAGCAGGACATCACCTCCCAGAGCCCAGCAGTAGGGAGACTG | |
| GCCGAGGTGCCAAGGGTGAGCGAGGTAGCAAGAGCCACCCAAACGCCCATGCCCGAGGC | |
| AGAGTCGGGGGCCTTGGGGCTCAGGGACCTTCCGGAAGCAGCGAGTGGGAAGACGAACA | |
| GTCTGAGTATTCTGATATCCGGAGGTGAAATGAAAGGCCTGGCCACGAAATCTTTCCTC | |
| CACGCCGTCCATTTTCTTATCTATGGACATTCCAAAACATTTACCATTAGAGAGGGGGG | |
| ATGTCACACGCAGGATTCTGTGGGGACTGTGGACTTCATCGAGGTGTGTGTTCGCGGAA | |
| CGGACAGGTGAGATGGAGACCCCTGGGGCCGTGGGGTCTCAGGGGTGCCTGGTGAATTC | |
| TGCACTTACACGTACTCAAGGGAGCGCGCCCGCGTTATCCTCGTACCTTTGTCTTCTTT | |
| CCATCTGTGGAGTCAGTGGGTGTCGGCCGCTCTGTTGTGGGGGAGGTGAACCAGGGAGG | |
| GGCAGGGCAAGGCAGGGCCCCCAGAGCTGGGCCACACAGTGGGTGCTGGGCCTCGCCCC | |
| GAAGCTTCTGGTGCAGCAGCCTCTGGTGCTGTCTCCGCGGAAGTCAGGGCGGCTGGATT | |
| CCAGGACAGGAGTGAATGTAAAAATAAATATCGCTTAGAATGCAGGAGAAGGGTGGAGA | |
| GGAGGCAGGGGCCGAGGGGGTGCTTGGTGCCAAACTGAAATTCAGTTTCTTGTGTGGGG | |
| CCTTGCGGTTCAGAGCTCTTGGCGAGGGTGGAGGGAGGAGTGTCATTTCTATGTGTAAT | |
| TTCTGAGCCATTGTACTGTCTGGGCTGGGGGGGACACTGTCCAAGGGAGTGGCCCCTAT | |
| GAGTTTATATTTTAACCACTGCTTCAAATCTCGATTTCACTTTTTTTATTTATCCAGTT | |
| ATATCTACATATCTGTCATCTAAATAAATGGCTTTCAAACAAAAAAAAAAAAAAAAAAA | |
| AAAAAAAA | |
| 302 amino acid reading frame | |
| MCAERLGQFMTLALVLATFDPARGTDATNPPEGPQDRSSQQKGRLSLQNTAEIQHCLVN | (Seq Id No. 4) |
| AGDVGCGVFECFENNSCEIRGLHGICMTFLHNAGKFDAQGKSFIKDALKCKAHALRHRF | |
| GCISRKCPAIREMVSQLQRECYLKHDLCAAAQENTRVIVEMIHFKDLLLHEPYVDLVNL | |
| LLTCGEEVKEAITHSVQVQCEQNWGSLCSILSFCTSAIQKPPTAPPERQPQVDRTKLSR | |
| AHHGEAGHHLPEPSSRETGRGAKGERGSKSHPNAHARGRVGGLGAQGPSGSSEWEDEQS | |
| EYSDIRR | |

OA2 includes an open reading frame from nucleotides 123 to 1028. The open reading frame encodes a polypeptide of 302 amino acids (SEQ ID NO:4). OA2 (STC2) contains 4 exons and the exon-intron boundaries are completely conserved between STC2 and stanniocalcin (STC1).

U.S. Pat. No. 6,171,822-B1 discloses the sequence of OA2 describing the, use of this sequence in disorders associated with, caused by or resulting in, changes in calcium, phosphate, magnesium, zinc and copper levels.

The human stanniocalcin-2 gene, OA2, has been mapped to 5q35.2.

OA2 is also useful for modulating electrolytic pathological conditions in bone, heart, kidney, pancreas and the vascular system, bone diseases, hypertension, renal failure, hyperthyroidism, hyperparathyroidism, certain carcinomas, sarcoidosis, pancreatitis and drug induced disorders such as hypercalcaemia. The predicted 302-amino acid OA2 protein shares 34% identity with STC1, GENBANK-ID U25997, [OMIM: 603665].

In another embodiment, the stanniocalcin-2 gene, OA2, slows-down or reverses some of the damage caused in OA by abnormal mineral deposition in Chondrocalcinosis, familial articular. Familial articular chondrocalcinosis is a chronic articular disease characterized by acute intermittent attacks of arthritis; the presence of calcium hypophosphate crystals in synovial fluid, cartilage and periarticular soft tissue; and, by x-ray, evidence of calcium deposition in articular cartilage. Chondrocalcinosis [OMIM: 118600] occurs in 3 forms: a hereditary form; a form associated with metabolic disorders such as hyperparathyroidism, hemochromatosis, hypothyroidism and Wilson disease; and a sporadic form, which may in some cases represent the hereditary form. Under the designation of chondrocalcinosis articularis, Aschoff et al. (1966) described a family with 4 affected persons in 2 generations. The disorder was manifested clinically by episodic inflammatory involvement, acute or subacute, of one or more joints. Calcified hyaline and fibrous cartilage is demonstrable by x-ray, particularly in large joints. In articular cartilage a dense narrow band follows the contour of the epiphysis. OA2 can also be used to treat craniometaphyseal dysplasia [OMIM 605145].

Another equally important action of human stanniocalcin-2, OA2, is stimulation of phosphate reabsorption by renal proximal tubules. The consequence of this renal effect is increased levels of plasma phosphate, which combines with excess calcium and promotes its disposal into bone.

Considering the potential role of stanniocalcin in calcium phosphate homeostasis, treatment with human stanniocalcin-2 applied as a protein therapeutic can slow the progression of OA and may act at the level of the subchondral bone defect observed in OA. Accordingly, an inflammatory pathology in a subject can be treated by administering to the subject a compsoition comprising an OA2 polypeptide. The inflammatory pathology to be treated includes osteoarthritis, and inflammation of the joint and joint space, and degeneration of the cartilage matrix. promoting repair of the cartilage matrix (i.e., to promote the synthesis and deposition of oligomatrix support proteins) and for promoting chondrocyte growth and development and reduce cellular apoptosis of chondrocyte. Additional disorders wherein the associated expression of OAX in inflammatory conditions include those that mediate pro-inflammatory events (Crohns disease and ulcerative colitis), inflammatory lung disorders (asthma, chronic obstructive pulmonary disease (COPD), cystic fibrosis (CF), bronchiectasis and interstitial lung diseases) as well as Sjogren's syndrome, psoriasis, and rheumatoid arthritis.

A fragment of the human Stanniocalcin-2 gene (GenBank ID #AF055460 and confirmed via a fragment identified for SeqCalling ID sch_gb-af098462_{—}1) was initially found to be up-regulated in normal cartilage relative to cartilage from osteoarthritic knees using CuraGen's GeneCalling™ method of differential gene expression (see Example 1, infra). Sch_gb-af098462_{—}1 was confirmed as identical to the full length sequence for the human stanniocalcin gene (gbh_AF055460).

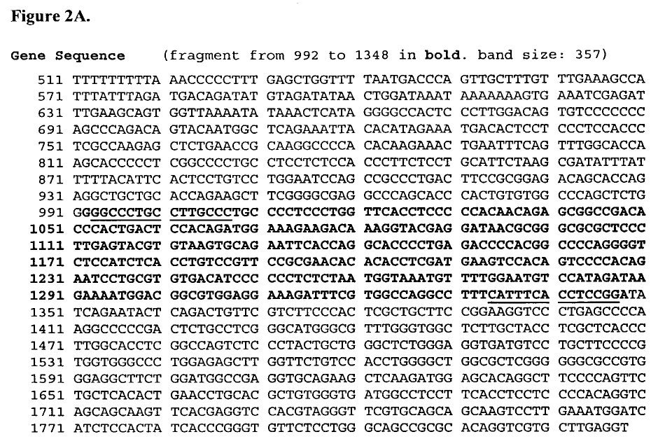

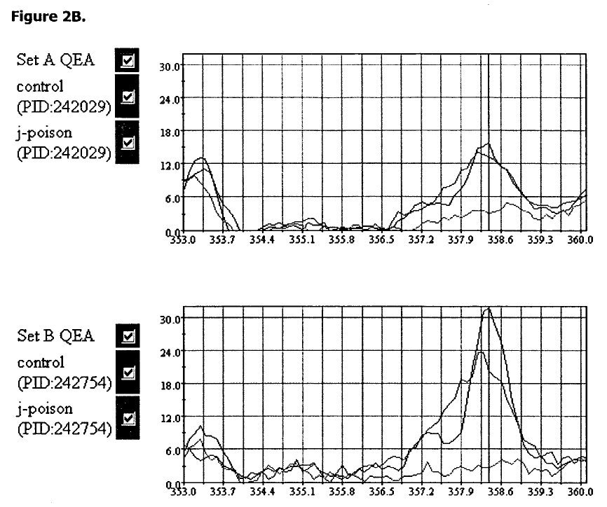

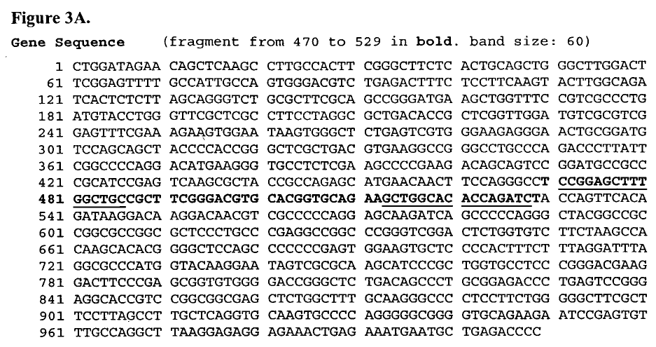

A differentially expressed human gene fragment migrating at approximately 358.4 nucleotides (nt) in length (FIG. 2B.—solid vertical line) was identified as a component of the human Stanniocalcin-2 cDNA (in the graphs, the abscissa is measured in lengths of nt and the ordinate is measured as signal response. Refer to traces in FIG. 1 (“QEA” serves as the original trace and “control” shows the recapitulation of the QEA following an independent chemistry reaction). Peak height differences between set A and B is used to calculate the N-fold difference is expression. The method of comparative PCR was used for confirmation of the gene assessment. The electropherographic peaks corresponding to the gene fragment of the human Stanniocalcin-2 are ablated when a gene-specific primer (see FIG. 2A,) competes with primers in the linker-adaptors during the PCR amplification. The peaks at 358.4 nt in length are ablated in the samples of human cartilage from normal (Set B) and osteoarthric (Set A) knees (refer to “r-poison” trace in FIG. 2B).

Taken together, these data show that Stanniocalcin-2 is down-regulated in osteoarthritic cartilage. Thus, treatment with Stanniocalcin-2 may be beneficial in the treatment of osteoarthritis and may help slow the progression of OA.

The OAX nucleic acids and polypeptides, as well as OAX antibodies, therapeutic agents and pharmaceutical compositions discussed herein, are useful, inter alia, in treating inflammatory conditions associated with osteoarthritis, inflammation of the joint and joint space, degeneration of the cartilage matrix, promoting repair of the cartilage matrix (i.e., to promote the synthesis and deposition of oligomatrix support proteins) and for promoting chondrocyte growth and development and reduce cellular apoptosis of chondrocyte.

OA3 Nucleic Acids and Polypeptides

A polynucleotide to be used with the methods of the present invention includes the nucleic acid sequence of OA3 (also refered to as Human adrenomedullin precursor or GenBank Accession No. D14874). The OA3 nucleic acid and encoded polypeptide sequences are shown in Table 3 (SEQ ID NO:5 and 6). The start and stop codons are shown in bold. The open reading frame (nucleotides 157 to 714 of SEQ ID NO:5) codes for a 370 amino acid long secreted protein (SEQ ID NO:6). OA3 has a predicted molecular weight of 42,808 daltons and a pI of 7.53. Protein structure analysis using PFAM and PROSITE identified the core PDGF domain within the OA3 polypeptide sequence.

TABLE 3
Nucleotide (SEQ ID NO:5) and Protein (SEQ ID NO:6) Sequence of OA3

| | |
|---|---|
| Sequence for gbh_d14874; cds = 157 to 714 bp | |
| CTGGATAGAACAGCTCAAGCCTTGCCACTTCGGGCTTCTCACTGCAGCTGGGCTTGGACTTCGGAGTTTTGCCATT | (Seq Id No. 5) |
| GCCAGTGGGACGTCTGAGACTTTCTCCTTCAAGTACTTGGCAGATCACTCTCTTAGCAGGGTCTGCGCTTCGCAGC | |
| CGGGATGAAGCTGGTTTCCGTCGCCCTGATGTACCTGGGTTCGCTCGCCTTCCTAGGCGCTGACACCGCTCGGTTG | |
| GATGTCGCGTCGGAGTTTCGAAAGAAGTGGAATAAGTGGGCTCTGAGTCGTGGGAAGAGGGAACTGCGGATGTCCA | |
| GCAGCTACCCCACCGGGCTCGCTGACGTGAAGGCCGGGCCTGCCCAGACCCTTATTCGGCCCCAGGACATGAAGGG | |
| TGCCTCTCGAAGCCCCGAAGACAGCAGTCCGGATGCCGCCCGCATCCGAGTCAAGCGCTACCGCCAGAGCATGAAC | |
| AACTTCCAGGGCCTCCGGAGCTTTGGCTGCCGCTTCGGGACGTGCACGGTGCAGAAGCTGGCACACCAGATCTACC | |
| AGTTCACAGATAAGGACAAGGACAACGTCGCCCCCAGGAGCAAGATCAGCCCCCAGGGCTACGGCCGCCGGCGCCG | |
| GCGCTCCCTGCCCGAGGCCGGCCCGGGTCGGACTCTGGTGTCTTCTAAGCCACAAGCACACGGGGCTCCAGCCCCC | |
| CCGAGTGGAAGTGCTCCCCACTTTCTTTAGGATTTAGGCGCCCATGGTACAAGGAATAGTCGCGCAAGCATCCCGC | |
| TGGTGCCTCCCGGGACGAAGGACTTCCCGAGCGGTGTGGGGACCGGGCTCTGACAGCCCTGCGGAGACCCTGAGTC | |
| CGGGAGGCACCGTCCGGCGGCGAGCTCTGGCTTTGCAAGGGCCCCTCCTTCTGGGGGCTTCGCTTCCTTAGCCTTG | |
| CTCAGGTGCAAGTGCCCCAGGGGGCGGGGTGCAGAAGAATCCGAGTGTTTGCCAGGCTTAAGGAGAGGAGAAACTG | |
| AGAAATGAATGCTGAGACCCCCGGAGCAGGGGTCTGAGCCACAGCCGTGCTCGCCCACAAACTGATTTCTCACGGC | |
| GTGTCACCCCACCAGGGCGCAAGCCTCACTATTACTTGAACTTTCCAAAACCTAAAGAGGAAAAGTGCAATGCGTG | |
| TTGTACATACAGAGGTAACTATCAATATTTAAGTTTGTTGCTGTCAAGATTTTTTTTGTAACTTCAAATATAGAGA | |
| TATTTTTGTACGTTATATATTGTATTAAGGGCATTTTAAAAGCAATTATATTGTCCTCCCCTATTTTAAGACGTGA | |
| ATGTCTCAGCGAGGTGTAAAGTTGTTCGCCGCGTGGAATGTGAGTGTGTTTGTGTGCATGAAAGAGAAAGACTGAT | |
| TACCTCCTGTGTGGAAGAAGGAAACACCGAGTCTCTGTATAATCTATTTACATAAAATGGGTGATATGCGAACAGC | |
| AAACC | |
| Frame: +1 - Nucleotide 157 to 711 | |
| MKLVSVALMYLGSLAFLGADTARLDVASEFRKKWNKWALSRGKRELRMSSSYPTGLADVKAGPAQTLIRPQDMKGA | (Seq Id No. 6) |
| SRSPEDSSPDAARIRVKRYRQSMNNFQGLRSFGCRFGTCTVQKLAHQIYQFTDKDKDNVAPRSKISPQGYGRRRRR | |
| SLPEAGPGRTLVSSKPQAHGAPAPPSGSAPHFL | |

The Human adrenomedullin gene, OA3, has been mapped to 5p14.2-q15. Recent evidence has shown that adrenomedullin may presynaptically inhibit neurotransmission of Calcitonin Gene-Related Peptide (CGRP)-ergic nerves, probably decreasing CGRP release, via receptors different from CGRP receptors [PMID: 11754977] [Akiyama S, et al. Peptides. November 2001; 22 (11):1887-93.]. CGRP is a potent vasodilator, which causes reduction in serum calcium. Likewise adrenomedullin (which is related to CGRP and binds to the same receptor) also acts as a vasodilator. CGRP has been shown to have a physiological and/or pathological role in neurogenic inflammation, in addition, blocking the action of CGRP inhibits neurogenic inflammation [PMID: 11436516, Iwasawa Y, Danjo T. Nippon Yakurigaku Zasshi. June 2001; 117 (6):387-93]. There is also evidence that nerves may respond to joint damage via pronounced changes in neuropeptide-like immunoreactivity found in the joint fluid. [PMID: 8868297 Appelgren A, Appelgren B, Carleson J, Kopp S, Theodorsson E. A model for experimentally induced temperomandibular joint arthritis in rats: effects of carrageenan on neuropeptide-like immunoreactivity. Neuropeptides. February 1996; 30(1):37-41. Evidence has also been demonstrated for a link between CGRP and TMJ joint arthritis [PMID: 7540832, Alstergren P, et al. Arch Oral Biol. February 1995; 40(2):127-35.]. See also PMID: 11299299 Bulling DG, et al. J. Neurochem. April 2001; 77(2):372-82, 7520139 [Arnalich F, et al. Neurosci Lett. Apr. 11, 1994; 170(2):251-4].

Knee synovium has an extensive neural network of both the somatic and autonomic nervous systems. An especially strong response for Substance P (SP) and CGRP was observed at the free nerve endings in pre-operative OA knees in humans. Postoperative incidence of SP-positive free nerve endings was reduced to 54% of the preoperative amount and the inflammation subsided in the medial region [PMID: 1447524, Saito T. Nippon Seikeigeka Gakkai Zasshi. September 1992; 66 (9):884-97.].

In addition, significant increases in CGRP mRNAs were observed in small-diameter L5 DRG neurones innervating Adjuvant-induced inflammed joints [PMID: 11299299 Bulling DG, et al. J. Neurochem. April 2001; 77 (2):372-82]. Neurogenic inflammation results from peripheral release of Substance P and neurokinin A.

In a similar manner, the peripheral release of neuropeptides in the synovium of OA knees may contribute to the inflammatory response in the joint space, further contributing to the progression of OA.

Adrenomedullin, OA3, has both apoptotic and antiapoptotic effects in various cells. In osteoarthritis, chondrocyte proliferation plays a role in sustaining the production of proteins used for the rebuilding of cartilage matrix during high matrix turnover. Therefore, Adrenomedullin, OA3, may mediate cellular growth of chondrocytes in OA damaged cartilage as a protein therapeutic, thereby promoting tissue repair and improving the condition of the joints. In a preferred embodiment, Adrenomedullin, OA3 can serve as a protein therapeutic to slow the progression of OA.

In another embodiment, Adrenomedullin (OA3) can be used as a protein therapeutic to decrease or inhibit the inflammation that contributes to the degradation of articular extracellular matrix. In addition adrenomedullin can be used to reduce the inflammation observed in the synovium that also occurs in rheumatoid arthritis.

A fragment of the human Adrenomedullin gene (GenBank ID #D14874 and confirmed via a fragment identified for the same accession number) was initially found to be up-regulated in normal cartilage relative to cartilage from osteoarthritic knees using CuraGen's GeneCalling™ method of differential gene expression (see Example 1, infra).

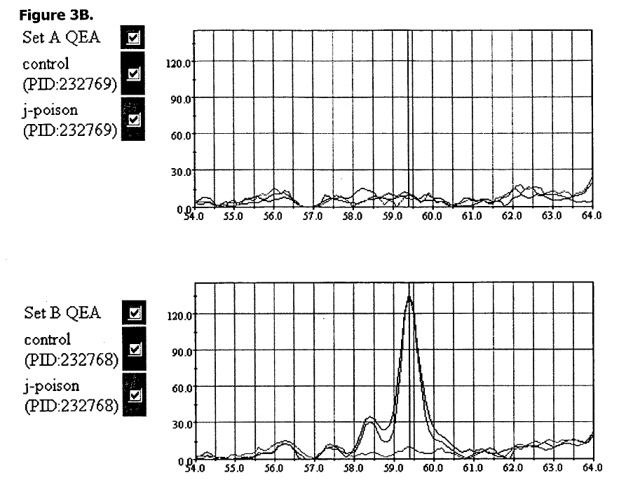

A differentially expressed human gene fragment migrating at approximately 59.4 nucleotides (nt) in length (FIG. 3B.—solid vertical line) was definitively identified as a component of the human Adrenomedullin cDNA (in the graphs, the abscissa is measured in lengths of nt and the ordinate is measured as signal response. Refer to traces in FIG. 1 (“QEA” serves as the original trace and “control” shows the recapitulation of the QEA following an independent chemistry reaction). Peak height differences between set A and B is used to calculate the N-fold difference is expression. The method of comparative PCR was used for confirmation of the gene assessment. The electropherographic peaks corresponding to the gene fragment of the human Adrenomedullin are ablated when a gene-specific primer (see FIG. 3A,) competes with primers in the linker-adaptors during the PCR amplification. The peaks at 59.4 nt in length are ablated in the samples of human cartilage from normal (Set B) and osteoarthric (Set A) knees (refer to “r-poison” trace in FIG. 3B). Detectable expression of this gene was also detected by RTQ-PCR of samples from a number of tissues from osteoarthritic and rheumatoid arthritis patients.

Taken together, these data show that Adrenomedullin is down-regulated in osteoarthritic cartilage. Thus, treatment with Adrenomedullin applied as a protein therapeutic can be utilized in the treament of osteoarthritis and may help slow the progression of OA.

The OAX nucleic acids and polypeptides, as well as OAX antibodies, therapeutic agents and pharmaceutical compositions discussed herein, are useful, inter alia, in treating inflammatory conditions associated with osteoarthritis, inflammation of the joint and joint space, degeneration of the cartilage matrix, promoting repair of the cartilage matrix (i.e., to promote the synthesis and deposition of oligomatrix support proteins) and for promoting chondrocyte growth and development and reduce cellular apoptosis of chondrocyte.

OA4 Nucleic Acids and Polypeptides

A OA4 (also refered to as Human latent transforming growth factor-beta-binding protein-2 (LTBP-2) or GenBank Accession No. Z37976.1) nucleic acid and polypeptide to be used with methods of the present invention includes the nucleic acid and encoded polypeptide sequence shown in Table 4 (SEQ ID NO:7 and 8). The start and stop codons are shown in bold.

The LTBP-2 (OA4) gene encodes a 195 kDa protein containing 20 epidermal growth factor (EGF)-like repeats, three repeats containing eight cysteines, and one segment that appears to be a hybrid of the two. Single exons encode EGF repeats while the eight-cysteine repeats are encoded in two exons.

TABLE 4
Nucleotide (SEQ ID NO: 7) and Protein (SEQ ID NO: 8) Sequence of OA4

| | |
|---|---|
| Sequence for gbh_s82451; cds = 388 to 5853 bp. | |
| CCTCGCTCCCTCTCCGGTAATCAGGGGGCTGAGCTGTCCCTCCGAGGAGGGGGCCTGGTGTGGATAAAAGAGACGA | (Seq Id No. 7) |
| AAAAGCCGGGGGAGGTTTCCAAAAATAAAACCGTCCGGGTCCCCTTCAGACGGCTGCAGGCACAGGGAGGAGGCGC | |
| GAAGGTGCAGCAGCCGTGCGAGCCCAGCTGGAGTAGGAGCGCGGACTCGAGGCTCGGGGCGCGCAGCCCTCGTTCC | |
| GCCGAGAGCCGGGCCCCCAGTCGGCCGCTTCAGGGCCCCCTAGACTCAGAGAAGCTGGCCGCCGGGCGGGGCCGGG | |
| AGAACAGCCCGCGGGCGTCCAGCGTGCCGACCACAAAGCTCTTCGCGGTGCCCGCGCGCACCACTCTCCAGCCGCC | |
| CCGCGCCATGAGGCCGCGGACCAAAGCCCGCAGCCCGGGGCGCGCCCTGCGGAACCCCTGGAGAGGCTTCCTGCCG | |
| CTCACCCTGGCTCTCTTCGTGGGCGCGGGTCATGCCCAAAGGGACCCCGTAGGGAGATACGAGCCGGCTGGTGGAG | |
| ACGCGAATCGACTGCGGCGCCCTGGGGGCAGCTACCCGGCAGCGGCTGCAGCCAAGGTGTACAGTCTGTTCCGGGA | |
| GCAGGACGCGCCTGTCGCGGGCTTGCAGCCCGTGGAGCGGGCCCAGCCGGGCTGGGGGAGCCCCAGGAGGCCCACC | |
| GAGGCGGAGGCCAGGAGGCCGTCCCGCGCGCAGCAGTCGCGGCGTGTCCAGCCACCTGCGCAGACCCGGAGAAGCA | |
| CTCCCCTGGGCCAGCAGCAACCAGCACCCCGGACCCGGGCCGCGCCGGCTCTCCCACGCCTGGGGACCCCACAGCG | |
| GTCTGGGGCTGCGCCCCCAACCCCGCCGCGAGGGCCGCTCACGGGGAGCAACGTCTGCGGGGGACAGTGCTGCCCA | |
| GGATGGACAACAGCAAACAGCACCAACCACTGTATCAAACCCGTTTGCGAGCCGCCGTGCCAGAACCGGGGCTCCT | |
| CCAGCCGCCCGCAGCTCTGTGTCTGCCGCTCTGGTTTCCGTGGAGCCCGCTGCGAGGAGGTCATTCCCGATGAGGA | |
| ATTTGACCCCCAGAACTCCAGGCTGGCACCTCGACGCTGGGCCGAGCGTTCACCCAACCTGCGCAGGAGCAGTGCG | |
| GCTGGAGAGGGCACCTTGGCCAGAGCACAGCCGCCAGCACCACAGTCGCCGCCCGCACCACAGTCGCCACCAGCTG | |
| GCACCCTGAGTGGCCTCAGCCAGACCCACCCTTCCCAGCAGCACGTGGGGTTGTCCCGCACTGTCCGACTTCACCC | |
| GACTGCCACGGCCAGTAGCCAGCTCTCTTCCAACGCCCTGCCCCCGGGACCAGGCCTTGAGCAGAGAGATGGCACC | |
| CAACAGGCGGTACCTCTGGAGCACCCCTCATCCCCCTGGGGGCTGAACCTCACGGAGAAAATCAAGAAGATCAAGA | |
| TCGTCTTCACTCCCACCATCTGCAAGCAGACCTGTGCCCGTGGACACTGTGCCAACAGCTGTGAGAGGGGCGACAC | |
| CACCACCCTGTACAGCCAGGGCGCCCATGGGCACGATCCCAAGTCTGGCTTCCGCATCTATTTCTGCCAGATCCCC | |
| TGCCTGAACGGAGGCCGCTGCATCGGCAGGGACGAATGCTGGTGCCCCGCCAACTCCACCGGGAAGTTCTGCCACC | |
| TGCCTATCCCGCAGCCGGACAGGGAGCCTCCAGGGAGGGGGTCCCGCCCCAGGGCCTTGCTGGAAGCCCCACTGAA | |
| GCAGTCCACTTTCACACTGCCGCTCTCCAACCAGCTGGCCTCCGTGAACCCCTCCCTGGTGAAGGTGCACATTCAC | |
| CACCCACCCGAGGCCTCAGTGCAGATCCACCAGGTGGCCCAGGTGCGGGGCGGGGTGGAGGAGGCCCTAGTGGAGA | |
| ACAGCGTGGAGACCAGACCCCCGCCCTGGCTGCCTGCCAGCCCTGGCCACAGCCTCTGGGACAGCAACAACATCCC | |
| TGCTCGGTCTGGAGAGCCCCCTCGGCCACTGCCCCCAGCAGCACCCAGGCCTCGAGGACTGCTGGGCCGGTGTTAC | |
| CTGAACACTGTGAACGGACAGTGTGCCAACCCTCTGCTGGAGCTGACTACCCAGGAGGACTGCTGTGGCAGTGTGG | |
| GAGCCTTCTGGGGGGTGACTTTGTGTGCCCCATGCCCACCCAGACCAGCCTCCCCGGTGATTGAGAATGGCCAGCT | |
| GGAGTGTCCTCAGGGGTACAAGAGACTGAACCTCACTCACTGCCAAGATATCAACGAGTGCTTGACCCTGGGCCTG | |
| TGCAAGGACGCGGAGTGTGTGAATACCAGGGGCAGCTACCTGTGCACATGCAGACCTGGCCTCATGCTGGATCCAT | |
| CGCGGAGCCGCTGTGTGTCGGACAAGGCAATCTCCATGCTGCAGGGACTGTGCTACCGGTCGCTGCGGCCCGGCAC | |
| CTGCACCCTGCCTTTGGCCCAGCGGATCACCAAGCAGATATGCTGCTGCAGCCGCGTGGGCAAAGCATGGGGCAGC | |
| GAGTGTGAGAAATGCCCTCTGCCTGGCACAGAGGCCTTCAGAGAGATCTGCCCTGCCGGCCACGGCTACACCTACG | |
| CGAGCTCCGACATCCGCCTGTCCATGAGGAAAGCCGAGGAGGAGGAACTGGCAAGGCCCCCAAGGGAGCAAGGGCA | |
| GAGGAGCAGCGGGGCACTGCCCGGGCCAGCAGAGAGGCAGCCCCTCCGGGTCGTCACGGACACCTGGCTTGAGGCC | |
| GGGACCATCCCTGACAAGGGTGACTCTCAGGCTGGCCAGGTCACGACCAGTGTCACTCATGCACCTGCCTGGGTCA | |
| CAGGGAATGCCACAACCCCACCAATGCCTGAACAGGGGATTGCAGAGATACAGGAAGAACAAGTGACCCCCTCCAC | |
| CGATGTGCTGGTGACCCTGAGCACCCCAGGCATTGACAGATGCGCTGCTGGAGCCACCAACGTCTGTGGCCCTGGA | |
| ACCTGCGTGAACCTCCCCGATGGATACAGATGTGTCTGCAGCCCTGGCTACCAGCTGCACCCCAGCCAGGCCTACT | |
| GCACAGATGACAACGAGTGTCTGAGGGACCCCTGCAAGGGAAAAGGGCGCTGCATCAACCGCGTGGGGTCCTACTC | |
| CTGCTTCTGCTACCCTGGCTACACTCTGGCCACCTCAGGGGCGACACAGGAGTGTCAAGATATCAATGAGTGTGAG | |
| CAGCCAGGGGTGTGCAGCGGGGGGCAGTGCACCAACACCGAGGGCTCGTACCACTGCGAGTGTGATCAGGGCTACA | |
| TCATGGTCAGGAAAGGACACTGCCAAGATATCAACGAATGCCGTCACCCCGGTACCTGCCCTGATGGGAGATGCGT | |
| CAATTCCCCTGGCTCCTACACTTGTCTGGCCTGTGAGGAGGGCTACCGGGGCCAGAGTGGGAGCTGTGTAGATGTG | |
| AATGAGTGTCTGACTCCCGGGGTCTGTGCCCATGGAAAGTGCACCAACCTAGAAGGCTCCTTCAGATGCTCTTGTG | |
| AGCAGGGCTATGAGGTCACCTCAGATGAGAAGGGCTGCCAAGATGTGGATGAGTGTGCCAGCCGGGCCTCATGCCC | |
| CACAGGCCTCTGCCTCAACACGGAGGGCTCCTTCGCCTGCTCTGCCTGTGAGAACGGGTACTGGGTGAATGAAGAC | |
| GGCACTGCCTGTGAAGACCTAGATGAGTGTGCCTTCCCGCGAGTCTGCCCCTCCGGAGTCTGCACCAACACGGCTG | |
| GCTCCTTCTCCTGCAAGGACTGCGATGGGGGCTACCGGCCCAGCCCCCTGGGTGACTCCTGTGAAGATGTGGATGA | |
| ATGTGAAGACCCCCAGAGCAGCTGCCTGGGAGGCGAGTGCAAGAACACTGTGGGCTCCTACCAGTGCCTCTGTCCC | |
| CAGGGCTTCCAGCTGGCCAATGGCACCGTGTGTGAGGATGTGAATGAGTGCATGGGGGAGGAGCACTGCGCACCCC | |
| ACGGCGAGTGCCTCAACAGCCACGGGTCTTTCTTCTGTCTGTGCGCGCCTGGCTTCGTCAGCGCAGAGGGGGGCAC | |
| CAGCTGCCAGGATGTGGACGAGTGTGCCACCACAGACCCGTGTGTGGGAGGGCACTGTGTCAACACCGAGGGCTCC | |
| TTCAACTGTCTATGTGAGACTGGCTTCCAGCCCTCCCCAGAGAGTGGAGAGTGTGTGGATATTGACGAGTGTGAGG | |
| ACTATGGAGACCCGGTGTGTGGCACCTGGAAGTGTGAAAACAGCCCTGGCTCCTACCGCTGTGTTCTGGGCTGCCA | |
| GCCTGGCTTCCACATGGCCCCGAACGGAGACTGCATTGACATAGACGAGTGCGCCAACGACACCATGTGTGGCAGC | |
| CACGCCTTCTGTGACAACACTGATGGCTCCTTCCGCTGCCTCTGTGACCAGGGCTTCGAGATCTCTCCCTCAGGCT | |
| GGGACTGTGTGGATGTGAACGAGTGTGAQCTTATGCTGGCGGTATGTGGGGCCGCGCTCTGTGAGAACGTGGAGGG | |
| CTCCTTCCTGTGCCTCTGTGCCAGTGACCTGGAGGAGTACGATGCCCAGGAGGGGCACTGCCGCCCACGGGGGGCT | |
| GGAGGTCAGAGTATGTCTGACGCCCCAACGGGGGACCATGCCCCGGCCCCCACCCGCATGGACTGCTACTCCGGGC | |
| AGAAGGGCCATGCGCCCTGCTCCAGTGTCCTGGGCCGGAACACCACACAGGCTGAATGCTGCTGCACCCAGGGCGC | |
| TAGCTGGGGAGATGCCTGTGACCTCTGCCCGTCTGAGGACTCAGCTGAATTCAGCGAGATCTGCCCTAGTGGAAAA | |
| GGCTACATTCCTGTGGAAGGAGCCTGGACGTTTGGACAGACCATGTACACAGATGCGGATGAGTGTGTGATATTCG | |
| GGCCTGGTCTCTGCCCGAACGGCCGGTGCCTCAACACCGTGCCTGGTTATGTCTGCCTGTGCAATCCCGGCTTCCA | |
| CTACGATGCTTCCCACAAGAAGTGTGAGGATCACGATGAGTGCCAGGACCTGGCCTGTGAGAATGGCGAGTGCGTC | |
| AACACGGAGGGCTCCTTCCACTGCTTCTGCAGCCCCCCGCTCACCCTGGACCTCAGCCAGCAGCGCTGCATGAACA | |
| GCACCAGCAGCACGGAGGACCTCCCTGACCACGACATCCACATGGACATCTGCTGGAAAAAAGTCACCAATGATGT | |
| GTGCAGCGAACCCCTGCGTGGGCACCGCACCACCTACACGGAATGCTGCTGCCAGGACGGCGAGGCCTGGAGCCAG | |
| CAGTGTGCTCTGTGTCCCCCGAGGAGCTCTGAGGTCTATGCTCAGCTGTGCAACGTGCCTCGCATTGAGGCAGAGC | |
| GGGAGGCCGGGGTCCACTTCCGGCCAGGCTATGAGTATGGCCCCGGGCCCGATGACCTGCACTACAGCATCTATGG | |
| CCCAGATGGGGCCCCCTTCTACAACTACCTGGGCCCCGAGGACACCGTCCCTGAGCCTGCCTTCCCCAACACAGCC | |
| GGTCACTCAGCGGACCGCACACCCATCCTTGAGTCTCCTTTGCAGCCCTCAGAACTCCAGCCCCACTACGTGGCCA | |
| GCCATCCAGAGCCCCCAGCCGGCTTCGAAGGGCTTCAGGCGGAGGAGTGCGGCATCCTGAACGGCTGTGAGAATGG | |
| CCGCTGTGTGCGCGTGCGGGAGGGCTACACCTGTGACTGTTTTGAGGGCTTCCAGCTGGATGCGGCCCACATGGCC | |
| TGCGTAGATGTGAATGAGTGTGATGACTTGAACGGGCCTGCTGTGCTCTGTGTCCATGGTTACTGCGAGAACACAG | |
| AGGGCTCCTACCGCTGCCACTGCTCCCCGGGATATGTCGCTGAGGCAGGGCCCCCCCACTGCACTGCCAAGGAGTA | |
| GCAGTCAGGGGTCAGTGTGGCAACTACCTGGAAATGGCCTCCAGTCACAGGCAGGGGCCTTGAGGATGATTTCCTA | |
| GCTGGGAAGACACCGTGACATCAGGCCAGAGGTTTCCAATCAGCCTTGCCTGCTTTCATCTCTCCCAGCTTAGCCT | |
| CTGGCTGTAAGCTTCGGTCATTGCCTCCATGCCCTTGCTTGGCTCAAGCACCACCAATCGCTTTAATGCTTCAGCC | |
| ACCGCATGAGGCCCTGTCCACCACCTTTCCTGGCCTTGCTATGGGATGCTTACCAAAGGATGGCCCTCATCCACCC | |
| TCCCAAGCTGTGCGAGCATGCAAGGCCCCATGGCCTCACACTGCAGACACCCCTTTCCAGCCACAATCCACCATCA | |
| TCCTGACGATCCCACAACTGGGACAGAGGCTACATCTGCCCTAGGGAGGTCCTTCAGAATCTGTGGAGCAAGAAAG | |
| GATTTGGGGAAGCTTGGGGACTGACTCCAGAGCCCCCTCCTAAGAACCATCACCACCACTCAGCCAATCTGTTCTG | |
| GGCCCTGATTTTGCCACACCTCCATCCTGTAGCCCATTCTCTGACCCCAAGGAGTGGCAGAAGATCCCTTCACTCA | |
| GAGAAGCAAGGCTGATATTAGCTTGTTGAATGTAAGAGACACAAATGAAGAAGAACAAAGAGCCTGAGAAAGCAGC | |
| AAGAGGACATGATGAAAAATACGTGGAGTTGATGAGAAAGGGGAGCCAAGGCTTTATACGTCTAAAGAAAATATTC | |
| AGTAGCTGAATCCCCCCAGTGATAGCCTGTGGGCACCAGCAGCAAGGGCTGCCATGGGATACAGCACCCATCTACA | |
| AAGACCTCTATTACATAAACACTGCTTCTTACAGGAAACAAACCTCTTCTGGGATCTCCTTTTGTGAAAACCAGTT | |
| TGATGTGCTAAAAGTAAAAAGTCTATTTTCCAGTGTGGTCTTGTTCAGAAGCAGCCAGATTTCCAATGTTGTTTTT | |
| CCCCTCCACTCAGAAACCCCTGCCCTTTCCCTTCAGAAAACGATGGCAGGCATTCCTCTGAGTTTACAAGCAGAGA | |
| CTCACTCCAACCCAAACTAGCTGGGAGTTCAGAACCATGGTGGAATAAAGAAATGTGCATCTGGTCCAAAAAAAAA | |
| AAAAAAAAAAAAAAAAAAAAAAAAA | |
| Protein Translation: | |
| Frame: +1 - Nucleotide 388 to 5850 - 1821 amino acid reading frame | |
| MRPRTKARSPGRALRNPWRGFLPLTLALFVGAGHAQRDPVGRYEPAGGDANRLRRPGGSYPAAAAAKVYSLFREQD | (Seq Id No. 8) |
| APVAGLQPVERAQPGWGSPRRPTEAEARRPSRAQQSRRVQPPAQTRRSTPLGQQQPAPRTRAAPALPRLGTPQRSG | |
| AAPPTPPRGRLTGRNVCGGQCCPGWTTANSTNHCIKPVCEPPCQNRGSCSRPQLCVCRSGFRGARCEEVIPDEEFD | |
| PQNSRLAPRRWAERSPNLRRSSAAGEGTLARAQPPAPQSPPAPQSPPAGTLSGLSQTHPSQQHVGLSRTVRLHPTA | |
| TASSQLSSNALPPGPGLEQRDGTQQAVPLEHPSSPWGLNLTEKIKKIKIVFTPTICKQTCARGHCANSCERGDTTT | |
| LYSQGGHGHDPKSGFRIYFCQIPCLNGGRCIGRDECWCPANSTGKFCHLPIPQPDREPPGRGSRPRALLEAPLKQS | |
| TFTLPLSNQLASVNPSLVKVHIHHPPEASVQIHQVAQVRGGVEEALVENSVETRPPPWLPASPGHSLWDSAAIPAR | |
| SGEPPRPLPPAAPRPRGLLGRCYLNTVNGQCANPLLELTTQEDCCGSVGAFWGVTLCAPCPPRPASPVIENGQLEC | |
| PQGYKRLNLTHCQDINECLTLGLCKDAECVNTRGSYLCTCRPGLMLDPSRSRCVSDKAISMLQGLCYRSLGPGTCT | |
| LPLAQRITKQICCCSRVGKAWGSECEKCPLPGTEAFREICPAGHGYTYASSDIRLSMRKAEEEELARPPREQGQRS | |
| SGALPGPAERQPLRVVTDTWLEAGTIPDKGDSQAGQVTTSVTHAPAWVTGNATTPPMPEQGIAEIQEEQVTPSTDV | |
| LVTLSTPGIDRCAAGATNVCGPGTCVNLPDGYRCVCSPGYQLHPSQAYCTDDNECLRDPCKGKGRCINRVGSYSCF | |
| CYPGYTLATSGATQECQDINECEQPGVCSGGQCTNTEGSYHCECDQGYIMVRKGHCQDINECRHPGTCPDGRCVNS | |
| PGSYTCLACEEGYRGQSGSCVDVNECLTPGVCAHGKCTNLEGSFRCSCEQGYEVTSDEKGCQDVDECASRASCPTG | |
| LCLNTEGSFACSACENGYWVNEDGTACEDLDECAFPGVCPSGVCTNTAGSFSCKDCDGGYRPSPLGDSCEDVDECE | |
| DPQSSCLGGECKNTVGSYQCLCPQGFQLANGTVCEDVNECMGEEHCAPHGECLNSHGSFFCLCAPGFVSAEGGTSC | |
| QDVDECATTDPCVGGHCVNTEGSFNCLCETGFQPSPESGECVDIDECEDYGDPVCGTWKCENSPGSYRCVLGCQPG | |
| FHMAPNGDCIDIDECANDTMCGSHGFCDNTDGSFRCLCDQGFEISPSGWDCVDVNECELMLAVCGAALCENVEGSF | |
| LCLCASDLEEYDAQEGHCRPRGAGGQSMSEAPTGDHAPAPTRMDCYSGQKGHAPCSSVLGRNTTQAECCCTQGASW | |
| GDACDLCPSEDSAEFSEICPSGKGYIPVEGAWTFCQTMYTDADECVIFGPGLCPNGRCLNTVPGYVCLCNPGFHYD | |
| ASHKKCEDHDECQDLACENGECVNTEGSFHCFCSPPLTLDLSQQRCMNSTSSTEDLPDHDIHMDICWKKVTNDVCS | |
| EPLRGHRTTYTECCCQDGEAWSQQCALCPPRSSEVYAQLCNVARIEAEREAGVHFRPGYEYGPGPDDLHYSIYGPD | |
| GAPFYNYLGPEDTVPEPAFPNTAGHSADRTPILESPLQPSELQPHYVASHPEPPAGFEGLQAEECGILNGCENGRC | |
| VRVREGYTCDCFEGFQLDAAHMACVDVNECDDLNGPAVLCVHGYCENTEGSYRCHCSPGYVAEAGPPHCTAKE | |

The human LTBP-2 gene (OA4) has been mapped to 14q24. Disorders mapped to this locus that are not already linked to a gene include Arrhythmogenic right ventricular dysplasia-1 [OMIM: 107970] Leber congenital amaurosis [OMIM: 604232] Cataract, anterior polar-1 [OMIM: 115650]

Transforming growth factor (TGF)-beta is secreted as an inactive complex, which frequently contains a large molecular weight binding protein designated latent TGF-beta-binding protein (LTBP). Latent transforming growth factor beta binding protein (LTBP), a high-molecular-weight glycoprotein of the large latent TGF-beta complex has been suggested to serve as an anchor for latent TGF-beta in the extracellular matrix and as a component of microfibrillar structures. Proteolytic cleavage of LTBP is supposed to be a prerequisite for the release and generation of bioactive (mature) TGF-beta. [Taipale J, et al. Adv Cancer Res. 1998;75:87-134].

Phylogenetic sequence comparisons demonstrated that LTBP-3 is more similar to LTBP-1 than LTBP-2, while LTBP-2 shows the most similarity to the fibrillins. Within the fibrillin family, fibrillin-1 is nearest to the LTBPs. While the domain structure of LTBP-2 is similar to that of the other LTBPs, LTBP-2 possesses unique regions that make it the largest member of the LTBP family. LTBP-2 may have dual functions as a member of the TGF-beta latent complex and as a structural component of microfibrils [PMID: 8697098, Bashir M M, et al. Int J Biochem Cell Biol. May 1996; 28 (5):531-42.].

The sequence for human latent transforming growth factor-beta-binding protein-2 (LTBP-2) gene (OA4) was down-regulated in knee cartilage from osteoarthritic subjects vs normal knee cartilage from control subjects. Treatment with LTBP-2 (OA4) applied as a protein therapeutic may help slow the progression of OA.

A fragment of the human LTBP-2 gene (GenBank ID #S82451 and confirmed via a fragment identified for the same accession number) was initially found to be up-regulated in normal cartilage relative to cartilage from osteoarthritic knees using CuraGen's GeneCalling™ method of differential gene expression (see Example 1, infra).

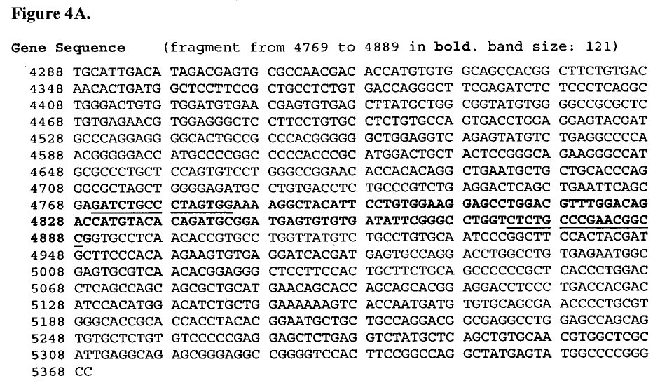

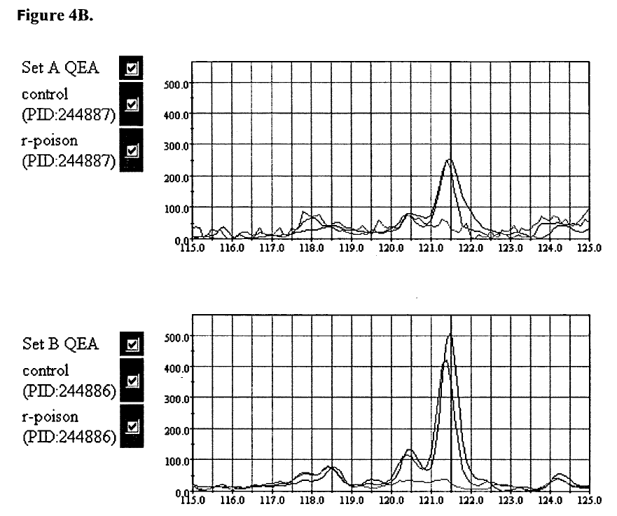

A differentially expressed human gene fragment migrating at approximately 121.5 nucleotides (nt) in length (FIG. 4B.—solid vertical line) was identified as a component of the human LTBP-2 cDNA (in the graphs, the abscissa is measured in lengths of nt and the ordinate is measured as signal response. Refer to traces in FIG. 1 (“QEA” serves as the original trace and “control” shows the recapitulation of the QEA following an independent chemistry reaction). Peak height differences between set A and B is used to calculate the N-fold difference is expression. The method of comparative PCR was used for confirmation of the gene assessment. The electropherographic peaks corresponding to the gene fragment of the human LTBP-2 are ablated when a gene-specific primer (see FIG. 4A,) competes with primers in the linker-adaptors during the PCR amplification. The peaks at 121.5 nt in length are ablated in the samples of human cartilage from normal (Set B) and osteoarthric (Set A) knees (refer to “r-poison” trace in FIG. 4B). Detectable expression of this gene was also detected by RTQ-PCR of samples from a number of tissues from osteoarthritic patients.

Taken together, these data show that LTBP-2 is down-regulated in osteoarthritic cartilage. Thus, treatment with LTBP-2 applied as a protein therapeutic can be utilized in the treatment of osteoarthritis and may help slow the progression of OA.

The OAX nucleic acids and polypeptides, as well as OAX antibodies, therapeutic agents and pharmaceutical compositions discussed herein, are useful, inter alia, in treating inflammatory conditions associated with osteoarthritis, inflammation of the joint and joint space, degeneration of the cartilage matrix, promoting repair of the cartilage matrix (i.e., to promote the synthesis and deposition of oligomatrix support proteins) and for promoting chondrocyte growth and development and reduce cellular apoptosis of chondrocyte.

OAX Therapeutic Proteins

An OAX nucleic acid or gene product is useful as a therapeutic agent in promoting cartilage repair, reducing inflammation, promoting chondrocyte growth and development and reduce cellular apoptosis of chondrocyte. It is intended in such cases that administration of a OAX nucleic acid or polypeptide, e.g., a polypeptide including the amino acid sequences described herein, or a nucleic acid sequence encoding these polypeptides will be controlled in doses such that any adverse side effects are minimized.

Included within the invention are OAX nucleic acids, isolated nucleic acids that encode OAX polypeptides or a portion thereof, OAX polypeptides, vectors containing these nucleic acids, host cells transformed with the OAX nucleic acids, anti-OAX antibodies, and pharmaceutical compositions useful in the treatment of musculoskeletal conditions and disorders. Disclosed are methods of making OAX polypeptides, as well as methods of screening, diagnosing, treating conditions using these compounds, and methods of screening compounds that modulate OAX polypeptide activity. The OAX nucleic acids and polypeptides, as well as OAX antibodies, therapeutic agents and pharmaceutical compositions discussed herein, are useful, inter alia, in treating inflammatory conditions associated with osteoarthritis, inflammation of the joint and joint space, degeneration of the cartilage matrix, promoting repair of the cartilage matrix (i.e., to promote the synthesis and deposition of oligomatrix support proteins) and for promoting chondrocyte growth and development and reduce cellular apoptosis of chondrocyte.

One aspect of the invention pertains to isolated nucleic acid molecules that encode OAX polypeptides or biologically active portions thereof used in the treatment of musculoskeletal conditions and disorders. Also included in the invention are nucleic acid fragments and biologically active proteins and fragments thereof used as small molecule targets or antibody targets. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated OAX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOS:1, 3, 5, 7 or a complement of this aforementioned nucleotide sequence, can be isolated using standard molecular biology techniques and the sequence information provided herein. Using all or a portion of the nucleic acid sequence of SEQ ID NOS:1, 3, 5, 7 as a hybridization probe, OAX molecules can be isolated using standard hybridization and cloning techniques (e.g., as described in Sambrook, et al., (eds.), MOLECULAR CLONING: A LABORATORY MANUAL 2nd Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to OAX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS:1, 3, 5, 7 or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7 or a portion of this nucleotide sequence (e.g., a fragment that can be used as a probe or primer or a fragment encoding a biologically-active portion of an OAX polypeptide). A nucleic acid molecule that is complementary to the nucleotide sequence shown in SEQ ID NOS:1, 3, 5, 7 is one that is sufficiently complementary to the nucleotide sequence shown in SEQ ID NOS:1, 3, 5, 7, that it can hydrogen bond with little or no mismatches to the nucleotide sequence shown SEQ ID NOS:1, 3, 5, 7 thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of OAX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an OAX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human OAX protein. Homologous nucleic acid sequences include those nucleic acid sequences that encode conservative amino acid substitutions (see below) in SEQ ID NOS:1, 3, 5, 7 as well as a polypeptide possessing OAX biological activity. Various biological activities of the OAX proteins are described below.

As used herein, “identical” residues correspond to those residues in a comparison between two sequences where the equivalent nucleotide base or amino acid residue in an alignment of two sequences is the same residue. Residues are alternatively described as “similar” or “positive” when the comparisons between two sequences in an alignment show that residues in an equivalent position in a comparison are either the same amino acid or a conserved amino acid as defined below.

An OAX polypeptide is encoded by the open reading frame (“ORF”) of an OAX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human OAX genes allows for the generation of probes and primers designed for use in identifying and/or cloning OAX homologues in other cell types, e.g. from other tissues, as well as OAX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOS:1, 3, 5, 7 or an anti-sense strand nucleotide sequence of SEQ ID NOS:1, 3, 5, 7 of a naturally occurring mutant of SEQ ID NOS:1, 3, 5, 7.

Probes based on the human OAX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which misexpress an OAX protein, such as by measuring a level of an OAX-encoding nucleic acid in a sample of cells from a subject e.g., detecting OAX mRNA levels or determining whether a genomic OAX gene has been mutated or deleted.

“A polypeptide having a biologically-active portion of an OAX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of OAX” can be prepared by isolating a portion SEQ ID NOS:1, 3, 5, 7, that encodes a polypeptide having an OAX biological activity (the biological activities of the OAX proteins are described below), expressing the encoded portion of OAX protein (e.g., by recombinant expression in vitro) and assessing the activity of the encoded portion of OAX.

OAX Nucleic Acid and Polypeptide Variants

The invention further encompasses nucleic acid molecules that differ from the nucleotide sequences shown SEQ ID NOS:1, 3, 5, 7 due to degeneracy of the genetic code and thus encode the same OAX proteins as that encoded by the nucleotide sequences shown in SEQ ID NOS:1, 3, 5, 7. In another embodiment, an isolated nucleic acid molecule of the invention has a nucleotide sequence encoding a protein having an amino acid sequence shown in SEQ ID NOS:2, 4, 6, 8.

In addition to the human OAX nucleotide sequences shown in SEQ ID NOS:1, 3, 5, 7 it will be appreciated by those skilled in the art that DNA sequence polymorphisms that lead to changes in the amino acid sequences of the OAX polypeptides may exist within a population (e.g., the human population). Such genetic polymorphism in the OAX genes may exist among individuals within a population due to natural allelic variation. As used herein, the terms “gene” and “recombinant gene” refer to nucleic acid molecules comprising an open reading frame (ORF) encoding an OAX protein, preferably a vertebrate OAX protein. Such natural allelic variations can typically result in 1-5% variance in the nucleotide sequence of the OAX genes. Any and all such nucleotide variations and resulting amino acid polymorphisms in the OAX polypeptides, which are the result of natural allelic variation and that do not alter the functional activity of the OAX polypeptides, are intended to be within the scope of the invention.

Moreover, nucleic acid molecules encoding OAX proteins from other species, and thus that have a nucleotide sequence that differs from the human sequence SEQ ID NOS:1, 3, 5, 7, are intended to be within the scope of the invention. Nucleic acid molecules corresponding to natural allelic variants and homologues of the OAX cDNAs of the invention can be isolated based on their homology to the human OAX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding OAX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences of SEQ ID NOS:1, 3, 5, 7 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7 or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NOS:1, 3, 5, 7 or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of OAX sequences that may exist in the population, the skilled artisan will further appreciate that changes can be introduced by mutation into the nucleotide sequences of SEQ ID NOS:1, 3, 5, 7 thereby leading to changes in the amino acid sequences of the encoded OAX proteins, without altering the functional ability of said OAX proteins. For example, nucleotide substitutions leading to amino acid substitutions at “non-essential” amino acid residues can be made in the sequence of SEQ ID NOS:2, 4, 6, 8 a “non-essential” amino acid residue is a residue that can be altered from the wild-type sequences of the OAX proteins without altering their biological activity, whereas an “essential” amino acid residue is required for such biological activity. For example, amino acid residues that are conserved among the OAX proteins of the invention are predicted to be particularly non-amenable to alteration. Amino acids for which conservative substitutions can be made are well-known within the art.

Another aspect of the invention pertains to nucleic acid molecules encoding OAX proteins that contain changes in amino acid residues that are not essential for activity. Such OAX proteins differ in amino acid sequence from SEQ ID NOS:2, 4, 6, 8 yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NOS:2, 4, 6, 8 Preferably, the protein encoded by the nucleic acid molecule is at least about 60% homologous to SEQ ID NOS:2, 4, 6, 8; more preferably at least about 70% homologous to SEQ ID NOS:2, 4, 6, 8 still more preferably at least about 80% homologous to SEQ ID NOS:2, 4, 6, 8 even more preferably at least about 90% homologous to SEQ ID NOS:2, 4, 6, 8 and most preferably at least about 95% homologous to SEQ ID NOS:2, 4, 6, 8.

An isolated nucleic acid molecule encoding an OAX protein homologous to the protein of SEQ ID NOS:2, 4, 6, 8 can be created by introducing one or more nucleotide substitutions, additions or deletions into the nucleotide sequence of SEQ ID NOS:1, 3, 5, 7 such that one or more amino acid substitutions, additions or deletions are introduced into the encoded protein.

Mutations can be introduced into SEQ ID NOS:2, 4, 6, 8 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the OAX protein is replaced with another amino acid residue from the same side chain family. Alternatively, in another embodiment, mutations can be introduced randomly along all or part of an OAX coding sequence, such as by saturation mutagenesis, and the resultant mutants can be screened for OAX biological activity to identify mutants that retain activity. Following mutagenesis of SEQ ID NOS:1, 3, 5, 7, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant OAX protein can be assayed for (i) the ability to form protein:protein interactions with other OAX proteins, other cell-surface proteins, or biologically-active portions thereof, (ii) complex formation between a mutant OAX protein and an OAX ligand; or (iii) the ability of a mutant OAX protein to bind to an intracellular target protein or biologically-active portion thereof; (e.g. avidin proteins).

In yet another embodiment, a mutant OAX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7 or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire OAX coding strand, or to only a portion thereof. Nucleic acid molecules encoding fragments, homologs, derivatives and analogs of an OAX protein of SEQ ID NOS:2, 4, 6, 8, or antisense nucleic acids complementary to an OAX nucleic acid sequence of SEQ ID NOS:1, 3, 5, 7 are additionally provided.

In one embodiment, an antisense nucleic acid molecule is antisense to a “coding region” of the coding strand of a nucleotide sequence encoding an OAX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the OAX protein. The term “noncoding region” refers to 5′ and 3′ sequences which flank the coding region that are not translated into amino acids (i.e., also referred to as 5′ and 3′ untranslated regions).

Given the coding strand sequences encoding the OAX protein disclosed herein, antisense nucleic acids of the invention can be designed according to the rules of Watson and Crick or Hoogsteen base pairing. The antisense nucleic acid molecule can be complementary to the entire coding region of OAX mRNA, but more preferably is an oligonucleotide that is antisense to only a portion of the coding or noncoding region of OAX mRNA. For example, the antisense oligonucleotide can be complementary to the region surrounding the translation start site of OAX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyluracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, uracil-5-oxyacetic acid methylester, uracil-5-oxyacetic acid (v), 5-methyl-2-thiouracil, 2,6-diaminopurine, (acp3)w, and 3-(3-amino-3-N-2-carboxypropyl) uracil. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an OAX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual α-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave OAX mRNA transcripts to thereby inhibit translation of OAX mRNA. A ribozyme having specificity for an OAX-encoding nucleic acid can be designed based upon the nucleotide sequence of an OAX cDNA disclosed herein (i.e., SEQ ID NOS:1, 3, 5, 7). For example, a derivative of a Tetrahymena L-19 IVS RNA can be constructed in which the nucleotide sequence of the active site is complementary to the nucleotide sequence to be cleaved in an OAX-encoding mRNA. See, e.g., U.S. Pat. No. 4,987,071 to Cech, et al. and U.S. Pat. No. 5,116,742 to Cech, et al. OAX mRNA can also be used to select a catalytic RNA having a specific ribonuclease activity from a pool of RNA molecules. See, e.g., Bartel et al., (1993) Science 261:1411-1418.

Alternatively, OAX gene expression can be inhibited by targeting nucleotide sequences complementary to the regulatory region of the, OAX nucleic acid (e.g., the OAX promoter and/or enhancers) to form triple helical structures that prevent transcription of the OAX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the OAX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of OAX can be used in therapeutic and diagnostic applications. For example, PNAs can be used as antisense or antigene agents for sequence-specific modulation of gene expression by, e.g., inducing transcription or translation arrest or inhibiting replication. PNAs of OAX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of OAX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of OAX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

OAX Polypeptides

A polypeptide according to the invention includes a polypeptide including the amino acid sequence of OAX polypeptides whose sequences are provided in SEQ ID NOS:2, 4, 6, 8. The invention also includes a mutant or variant protein any of whose residues may be changed from the corresponding residues shown in SEQ ID NOS:2, 4, 6, 8 while still encoding a protein that maintains its OAX activities and physiological functions, or a functional fragment thereof.

In general, an OAX variant that preserves OAX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated OAX proteins, and biologically-active portions thereof, or derivatives, fragments, analogs or homologs thereof. Also provided are polypeptide fragments suitable for use as immunogens to raise anti-OAX antibodies. In one embodiment, native OAX proteins can be isolated from cells or tissue sources by an appropriate purification scheme using standard protein purification techniques. In another embodiment, OAX proteins are produced by recombinant DNA techniques. Alternative to recombinant expression, an OAX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the OAX protein is derived, or substantially free from chemical precursors or other chemicals when chemically synthesized. The language “substantially free of cellular material” includes preparations of OAX proteins in which the protein is separated from cellular components of the cells from which it is isolated or recombinantly-produced. In one embodiment, the language “substantially free of cellular material” includes preparations of OAX proteins having less than about 30% (by dry weight) of non-OAX proteins (also referred to herein as a “contaminating protein”), more preferably less than about 20% of non-OAX proteins, still more preferably less than about 10% of non-OAX proteins, and most preferably less than about 5% of non-OAX proteins. When the OAX protein or biologically-active portion thereof is recombinantly-produced, it is also preferably substantially free of culture medium, i.e., culture medium represents less than about 20%, more preferably less than about 10%, and most preferably less than about 5% of the volume of the OAX protein preparation.

The language “substantially free of chemical precursors or other chemicals” includes preparations of OAX proteins in which the protein is separated from chemical precursors or other chemicals that are involved in the synthesis of the protein. In one embodiment, the language “substantially free of chemical precursors or other chemicals” includes preparations of OAX proteins having less than about 30% (by dry weight) of chemical precursors or non-OAX chemicals, more preferably less than about 20% chemical precursors or non-OAX chemicals, still more preferably less than about 10% chemical precursors or non-OAX chemicals, and most preferably less than about 5% chemical precursors or non-OAX chemicals.

Biologically-active portions of OAX proteins include peptides comprising amino acid sequences sufficiently homologous to or derived from the amino acid sequences of the OAX proteins (e.g., the amino acid sequence shown in SEQ ID NOS:2, 4, 6, 8) that include fewer amino acids than the full-length OAX proteins, and exhibit at least one activity of an OAX protein. Typically, biologically-active portions comprise a domain or motif with at least one activity of the OAX protein. A biologically-active portion of an OAX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native OAX protein.

In an embodiment, the OAX protein has an amino acid sequence shown in SEQ ID NOS:2, 4, 6, 8. In other embodiments, the OAX protein is substantially homologous to SEQ ID NOS:2, 4, 6, 8 and retains the functional activity of the protein of SEQ ID NOS:2, 4, 6, 8 yet differs in amino acid sequence due to natural allelic variation or mutagenesis, as described in detail, below. Accordingly, in another embodiment, the OAX protein is a protein that comprises an amino acid sequence at least about 45% homologous to the amino acid sequence SEQ ID NOS:2, 4, 6, 8 and retains the functional activity of the OAX proteins of SEQ ID NOS:2, 4, 6, 8.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides OAX chimeric or fusion proteins. As used herein, an OAX “chimeric protein” or “fusion protein” comprises an OAX polypeptide operatively-linked to a non-OAX polypeptide. An “OAX polypeptide” refers to a polypeptide having an amino acid sequence corresponding to an OAX protein (SEQ ID NOS:2, 4, 6, 8), whereas a “non-OAX polypeptide” refers to a polypeptide having an amino acid sequence corresponding to a protein that is not substantially homologous to the OAX protein, e.g. a protein that is different from the OAX protein and that is derived from the same or a different organism. Within an OAX fusion protein the OAX polypeptide can correspond to all or a portion of an OAX protein. In one embodiment, an OAX fusion protein comprises at least one biologically-active portion of an OAX protein. In another embodiment, an OAX fusion protein comprises at least two biologically-active portions of an OAX protein. In yet another embodiment, an OAX fusion protein comprises at least three biologically-active portions of an OAX protein. Within the fusion protein, the term “operatively-linked” is intended to indicate that the OAX polypeptide and the non-OAX polypeptide are fused in-frame with one another. The non-OAX polypeptide can be fused to the N-terminus or C-terminus of the OAX polypeptide.

In one embodiment, the fusion protein is a GST-OAX fusion protein in which the OAX sequences are fused to the C-terminus of the GST (glutathione S-transferase) sequences. Such fusion proteins can facilitate the purification of recombinant OAX polypeptides.

In another embodiment, the fusion protein is an OAX protein containing a heterologous signal sequence at its N-terminus. In certain host cells (e.g., mammalian host cells), expression and/or secretion of OAX can be increased through use of a heterologous signal sequence.

In yet another embodiment, the fusion protein is an OAX-immunoglobulin fusion protein in which the OAX sequences are fused to sequences derived from a member of the immunoglobulin protein family. The OAX-immunoglobulin fusion proteins of the invention can be incorporated into pharmaceutical compositions and administered to a subject to inhibit an interaction between an OAX ligand and an OAX protein on the surface of a cell, to thereby suppress OAX-mediated signal transduction in vivo. The OAX-immunoglobulin fusion proteins can be used to affect the bioavailability of an OAX cognate ligand. Inhibition of the OAX ligand/OAX interaction may be useful therapeutically for both the treatment of proliferative and differentiative disorders, as well as modulating (e.g. promoting or inhibiting) cell survival. Moreover, the OAX-immunoglobulin fusion proteins of the invention can be used as immunogens to produce anti-OAX antibodies in a subject, to purify OAX ligands, and in screening assays to identify molecules that inhibit the interaction of OAX with an OAX ligand.

An OAX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An OAX-encoding nucleic acid can be cloned into such an expression vector such that the fusion moiety is linked in-frame to the OAX protein.

OAX Agonists and Antagonists

The invention also pertains to variants of the OAX proteins that function as either OAX agonists (i.e., mimetics) or as OAX antagonists. Variants of the OAX protein can be generated by mutagenesis (e.g., discrete point mutation or truncation of the OAX protein). An agonist of the OAX protein can retain substantially the same, or a subset of, the biological activities of the naturally occurring form of the OAX protein. An antagonist of the OAX protein can inhibit one or more of the activities of the naturally occurring form of the OAX protein by, for example, competitively binding to a downstream or upstream member of a cellular signaling cascade which includes the OAX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the OAX proteins. In one aspect, the OAX sequences described herein can be used as protein therapeutics to treat osteoarthritis. Particularly, one of skill in the art can utilize conventional methods to determine the expression of proteins in, e.g. arthritic tissue and non-arthritic tissue. As described below, through differential gene expression and RTQ-PCR data the sequences described herein are downregulated in various tissues associated with osteoarthritis. The administration of these sequences to a subject in need thereof can treat or ameliorate the conditions associated with musculoskeletal conditions, particularly osteoarthritis.

Variants of the OAX proteins that function as either OAX agonists (i.e., mimetics) or as OAX antagonists can be identified by screening combinatorial libraries of mutants (e.g., truncation mutants) of the OAX proteins for OAX protein agonist or antagonist activity. In one embodiment, a variegated library of OAX variants is generated by combinatorial mutagenesis at the nucleic acid level and is encoded by a variegated gene library. A variegated library of OAX variants can be produced by, for example, enzymatically ligating a mixture of synthetic oligonucleotides into gene sequences such that a degenerate set of potential OAX sequences is expressible as individual polypeptides, or alternatively, as a set of larger fusion proteins (e.g., for phage display) containing the set of OAX sequences therein. There are a variety of methods which can be used to produce libraries of potential OAX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential OAX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the OAX protein coding sequences can be used to generate a variegated population of OAX fragments for screening and subsequent selection of variants of an OAX protein. In one embodiment, a library of coding sequence fragments can be generated by treating a double stranded PCR fragment of an OAX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the OAX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of OAX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify OAX variants. See, e.g., Arkin and Yourvan, 1992. Proc. Natl. Acad. Sci. USA 89: 7811-7815; Delgrave, et al., 1993. Protein Engineering 6:327-331.

Anti-OAX Antibodies

Also included in the invention are antibodies to OAX proteins, or fragments of OAX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated OAX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation to neutralize proteins which are overexpressed in musculoskeletal tissues or to detect such proteins. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of OAX-related protein that is located on the surface of the protein, e.g., a hydrophilic region. A hydrophobicity analysis of the human OAX-related protein sequence will indicate which regions of a OAX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fe), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating musculoskeletal conditions. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-osteoarthritic activity can also be prepared using heterobifunctional cross-linkers as described in Wolff et al. Cancer Research, 53: 2560-2565 (1993). Alternatively, for example, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate). In one embodiment, such immunoconjugates are useful in targeting proteins which are overexpressed or are surmised to induce musculoskeletal conditions thereby inhibiting expression of the proteins.

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an OAX protein is facilitated by generation of hybridomas that bind to the fragment of an OAX protein possessing such a domain. Thus, antibodies that are specific for a desired domain within an OAX protein, or derivatives, fragments, analogs or homologs thereof, are also provided herein.

Anti-OAX antibodies may be used in methods known within the art relating to the localization and/or quantitation of an OAX protein (e.g., for use in measuring levels of the OAX protein within appropriate physiological samples, for use in diagnostic methods, for use in imaging the protein, and the like). In a given embodiment, antibodies for OAX proteins, or derivatives, fragments, analogs or homologs thereof, that contain the antibody derived binding domain, are utilized as pharmacologically-active compounds (hereinafter “Therapeutics”).

An anti-OAX antibody (e.g., monoclonal antibody) can be used to isolate an OAX polypeptide by standard techniques, such as affinity chromatography or immunoprecipitation. An anti-OAX antibody can facilitate the purification of natural OAX polypeptide from cells and of recombinantly-produced OAX polypeptide expressed in host cells. Moreover, an anti-OAX antibody can be used to detect OAX protein (e.g., in a cellular lysate or cell supernatant) in order to evaluate the abundance and pattern of expression of the OAX protein. Anti-OAX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

OAX Recombinant Expression Vectors and Host Cells

Another aspect of the invention pertains to vectors, preferably expression vectors, containing a nucleic acid encoding an OAX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g. in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., OAX proteins, mutant forms of OAX proteins, fusion proteins, etc.).

The recombinant expression vectors of the invention can be designed for expression of OAX proteins in prokaryotic or eukaryotic cells. For example, OAX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the OAX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, OAX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to OAX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, OAX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding OAX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) OAX protein. Accordingly, the invention further provides methods for producing OAX protein using the host cells of the invention. In one embodiment, the method comprises culturing the host cell of invention (into which a recombinant expression vector encoding OAX protein has been introduced) in a suitable medium such that OAX protein is produced. In another embodiment, the method further comprises isolating OAX protein from the medium or the host cell.

Transgenic OAX Animals

The host cells of the invention can also be used to produce non-human transgenic animals. For example, in one embodiment, a host cell of the invention is a fertilized oocyte or an embryonic stem cell into which OAX protein-coding sequences have been introduced. Such host cells can then be used to create non-human transgenic animals in which exogenous OAX sequences have been introduced into their genome or homologous recombinant animals in which endogenous OAX sequences have been altered. Such animals are useful for studying the function and/or activity of OAX protein and for identifying and/or evaluating modulators of OAX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous OAX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing OAX-encoding nucleic acid into the male pronuclei of a fertilized oocyte (e.g. by microinjection, retroviral infection) and allowing the oocyte to develop in a pseudopregnant female foster animal. The human OAX cDNA sequences of SEQ ID NOS:1, 3, 5, 7 can be introduced as a transgene into the genome of a non-human animal. Alternatively, a non-human homologue of the human OAX gene, such as a mouse OAX gene, can be isolated based on hybridization to the human OAX cDNA (described further supra) and used as a transgene. Intronic sequences and polyadenylation signals can also be included in the transgene to increase the efficiency of expression of the transgene. A tissue-specific regulatory sequence(s) can be operably-linked to the OAX transgene to direct expression of OAX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the OAX transgene in its genome and/or expression of OAX mRNA in tissues or cells of the animals. A transgenic founder animal can then be used to breed additional animals carrying the transgene. Moreover, transgenic animals carrying a transgene-encoding OAX protein can further be bred to other transgenic animals carrying other transgenes.

To create a homologous recombinant animal, a vector is prepared which contains at least a portion of an OAX gene into which a deletion, addition or substitution has been introduced to thereby alter, e.g., functionally disrupt, the OAX gene. The OAX gene can be a human gene (e.g., the cDNA of SEQ ID NOS:1, 3, 5, 7), but more preferably, is a non-human homologue of a human OAX gene. For example, a mouse homologue of human OAX gene of SEQ ID NOS:1, 3, 5, 7 can be used to construct a homologous recombination vector suitable for altering an endogenous OAX gene in the mouse genome. In one embodiment, the vector is designed such that, upon homologous recombination, the endogenous OAX gene is functionally disrupted (i.e., no longer encodes a functional protein; also referred to as a “knock out” vector).

Alternatively, the vector can be designed such that, upon homologous recombination, the endogenous OAX gene is mutated or otherwise altered but still encodes functional protein (e.g., the upstream regulatory region can be altered to thereby alter the expression of the endogenous OAX protein). In the homologous recombination vector, the altered portion of the OAX gene is flanked at its 5′- and 3′-termini by additional nucleic acid of the OAX gene to allow for homologous recombination to occur between the exogenous OAX gene carried by the vector and an endogenous OAX gene in an embryonic stem cell. The additional flanking OAX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced OAX gene has homologously-recombined with the endogenous OAX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The OAX nucleic acid molecules, OAX proteins, and anti-OAX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. Further, the OAX molecules described herein may be used in conjunction with know pharmaceutical compositions used to treat musculoskeletal conditions. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e g., an OAX protein or anti-OAX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express OAX protein (e.g., via a recombinant expression vector in a host cell in gene therapy applications), to detect OAX mRNA (e.g., in a biological sample) or a genetic lesion in an OAX gene, and to modulate OAX activity, as described further, below. In addition, the OAX proteins can be used to screen drugs or compounds that modulate the OAX protein activity or expression as well as to treat disorders characterized by insufficient or excessive production of OAX protein or production of OAX protein forms that have decreased or aberrant activity compared to OAX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-OAX antibodies of the invention can be used to detect and isolate OAX proteins and modulate OAX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to OAX proteins or have a stimulatory or inhibitory effect on, e.g., OAX protein expression or OAX protein activity. The invention also includes compounds identified in the screening assays described herein.

In one embodiment, the invention provides assays for screening candidate or test compounds which bind to or modulate the activity of the membrane-bound form of an OAX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of OAX protein, or a biologically-active portion thereof, on the cell surface is contacted with a test compound and the ability of the test compound to bind to an OAX protein determined. The cell, for example, can of mammalian origin or a yeast cell. Determining the ability of the test compound to bind to the OAX protein can be accomplished, for example, by coupling the test compound with a radioisotope or enzymatic label such that binding of the test compound to the OAX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of OAX protein, or a biologically-active portion thereof, on the cell surface with a known compound which binds OAX to form an assay mixture, contacting the assay mixture with a test compound, and determining the ability of the test compound to interact with an OAX protein, wherein determining the ability of the test compound to interact with an OAX protein comprises determining the ability of the test compound to preferentially bind to OAX protein or a biologically-active portion thereof as compared to the known compound.

In another embodiment, an assay is a cell-based assay comprising contacting a cell expressing a membrane-bound form of OAX protein, or a biologically-active portion thereof, on the cell surface with a test compound and determining the ability of the test compound to modulate (e.g., stimulate or inhibit) the activity of the OAX protein or biologically-active portion thereof. Determining the ability of the test compound to modulate the activity of OAX or a biologically-active portion thereof can be accomplished, for example, by determining the ability of the OAX protein to bind to or interact with an OAX target molecule. As used herein, a “target molecule” is a molecule with which an OAX protein binds or interacts in nature, for example, a molecule on the surface of a cell which expresses an OAX interacting protein, a molecule on the surface of a second cell, a molecule in the extracellular milieu, a molecule associated with the internal surface of a cell membrane or a cytoplasmic molecule. An OAX target molecule can be a non-OAX molecule or an OAX protein or polypeptide of the invention. In one embodiment, an OAX target molecule is a component of a signal transduction pathway that facilitates transduction of an extracellular signal (e.g. a signal generated by binding of a compound to a membrane-bound OAX molecule) through the cell membrane and into the cell. The target, for example, can be a second intercellular protein that has catalytic activity or a protein that facilitates the association of downstream signaling molecules with OAX.

Determining the ability of the OAX protein to bind to or interact with an OAX target molecule can be accomplished by one of the methods described above for determining direct binding. In one embodiment, determining the ability of the OAX protein to bind to or interact with an OAX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an OAX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an OAX protein or biologically-active portion thereof with a test compound and determining the ability of the test compound to bind to the OAX protein or biologically-active portion thereof. Binding of the test compound to the OAX protein can be determined either directly or indirectly as described above. In one such embodiment, the assay comprises contacting the OAX protein or biologically-active portion thereof with a known compound which binds OAX to form an assay mixture, contacting the assay mixture with a test compound, and determining the ability of the test compound to interact with an OAX protein, wherein determining the ability of the test compound to interact with an OAX protein comprises determining the ability of the test compound to preferentially bind to OAX or biologically-active portion thereof as compared to the known compound.

In still another embodiment, an assay is a cell-free assay comprising contacting OAX protein or biologically-active portion thereof with a test compound and determining the ability of the test compound to modulate (e.g. stimulate or inhibit) the activity of the OAX protein or biologically-active portion thereof. Determining the ability of the test compound to modulate the activity of OAX can be accomplished, for example, by determining the ability of the OAX protein to bind to an OAX target molecule by one of the methods described above for determining direct binding. In an alternative embodiment, determining the ability of the test compound to modulate the activity of OAX protein can be accomplished by determining the ability of the OAX protein further modulate an OAX target molecule. For example, the catalytic/enzymatic activity of the target molecule on an appropriate substrate can be determined as described, supra.

In yet another embodiment, the cell-free assay comprises contacting the OAX protein or biologically-active portion thereof with a known compound which binds OAX protein to form an assay mixture, contacting the assay mixture with a test compound, and determining the ability of the test compound to interact with an OAX protein, wherein determining the ability of the test compound to interact with an OAX protein comprises determining the ability of the OAX protein to preferentially bind to or modulate the activity of an OAX target molecule.

The cell-free assays of the invention are amenable to use of both the soluble form or the membrane-bound form of OAX protein. In the case of cell-free assays comprising the membrane-bound form of OAX protein, it may be desirable to utilize a solubilizing agent such that the membrane-bound form of OAX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either OAX protein or its target molecule to facilitate separation of complexed from uncomplexed forms of one or both of the proteins, as well as to accommodate automation of the assay. Binding of a test compound to OAX protein, or interaction of OAX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-OAX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or OAX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of OAX protein binding or activity determined using standard techniques.

Other techniques for immobilizing proteins on matrices can also be used in the screening assays of the invention. For example, either the OAX protein or its target molecule can be immobilized utilizing conjugation of biotin and streptavidin. Biotinylated OAX protein or target molecules can be prepared from biotin-NHS(N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with OAX protein or target molecules, but which do not interfere with binding of the OAX protein to its target molecule, can be derivatized to the wells of the plate, and unbound target or OAX protein trapped in the wells by antibody conjugation. Methods for detecting such complexes, in addition to those described above for the GST-immobilized complexes, include immunodetection of complexes using antibodies reactive with the OAX protein or target molecule, as well as enzyme-linked assays that rely on detecting an enzymatic activity associated with the OAX protein or target molecule.

In another embodiment, modulators of OAX protein expression are identified in a method wherein a cell is contacted with a candidate compound and the expression of OAX mRNA or protein in the cell is determined. The level of expression of OAX mRNA or protein in the presence of the candidate compound is compared to the level of expression of OAX mRNA or protein in the absence of the candidate compound. The candidate compound can then be identified as a modulator of OAX mRNA or protein expression based upon this comparison. For example, when expression of OAX mRNA or protein is greater (i.e., statistically significantly greater) in the presence of the candidate compound than in its absence, the candidate compound is identified as a stimulator of OAX mRNA or protein expression. Alternatively, when expression of OAX mRNA or protein is less (statistically significantly less) in the presence of the candidate compound than in its absence, the candidate compound is identified as an inhibitor of OAX mRNA or protein expression. The level of OAX mRNA or protein expression in the cells can be determined by methods described herein for detecting OAX mRNA or protein.

In yet another aspect of the invention, the OAX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with OAX (“OAX-binding proteins” or “OAX-bp”) and modulate OAX activity. Such OAX-binding proteins are also likely to be involved in the propagation of signals by the OAX proteins as, for example, upstream or downstream elements of the OAX pathway.

The two-hybrid system is based on the modular nature of most transcription factors, which consist of separable DNA-binding and activation domains. Briefly, the assay utilizes two different DNA constructs. In one construct, the gene that codes for OAX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an OAX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with OAX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the OAX sequences, SEQ ID NOS:1, 3, 5, 7 or fragments or derivatives thereof, can be used to map the location of the OAX genes, respectively, on a chromosome. The mapping of the OAX sequences to chromosomes is an important first step in correlating these sequences with genes associated with disease. The mapped OAX sequences may be useful in treating not only musculoskeletal conditions or disorders but also other diseases mapped to the chromosome on which such sequence was associated.

Briefly, OAX genes can be mapped to chromosomes by preparing PCR primers (preferably 15-25 bp in length) from the OAX sequences. Computer analysis of the OAX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the OAX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the OAX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the OAX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The OAX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the OAX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The OAX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining OAX protein and/or nucleic acid expression as well as OAX activity, in the context of a biological sample (e.g., blood, serum, cells, tissue) to thereby determine whether an individual is afflicted with a disease or disorder, or is at risk of developing a disorder, associated with aberrant OAX expression or activity, aberrant OAX expression or activity, or both. The disorders include pathology such as inflammatory conditions, musculoskeletal conditions or disorders including but not limited to osteoarthritis, inflammation of the joint and joint space, and degeneration of the cartilage matrix. In addition, OAX nucleic acids and their encoded polypeptides will be therapeutically useful for the prevention of osteoarthritis and the acceleration of the cartilage matrix repair (i.e., to promote the synthesis and deposition of oligomatrix support proteins) and for promoting chondrocyte growth and development and reduce cellular apoptosis of chondrocyte through gene therapy.

The invention also provides for prognostic (or predictive) assays for determining whether an individual is at risk of developing a disorder associated with OAX protein, nucleic acid expression or activity. For example, mutations or dysregulation in an OAX gene can be assayed in a biological sample. Such assays can be used for prognostic or predictive purpose to thereby prophylactically treat an individual prior to the onset of a disorder characterized by or associated with OAX protein, nucleic acid expression, or biological activity.

Another aspect of the invention provides methods for determining OAX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of OAX in clinical trials.

These and other agents are described in further detail in the following sections.

Diagnostic Assays

An exemplary method for detecting the presence or absence of OAX in a biological sample involves obtaining a biological sample from a test subject and contacting the biological sample with a compound or an agent capable of detecting OAX protein or nucleic acid (e.g., mRNA, genomic DNA) that encodes OAX protein such that the presence of OAX is detected in the biological sample. An agent for detecting OAX mRNA or genomic DNA is a labeled nucleic acid probe capable of hybridizing to OAX mRNA or genomic DNA. The nucleic acid probe can be, for example, a full-length OAX nucleic acid, such as the nucleic acid of SEQ ID NOS:1, 3, 5, 7 or a portion thereof, such as an oligonucleotide of at least 15, 30, 50, 100, 250 or 500 nucleotides in length and sufficient to specifically hybridize under stringent conditions to OAX mRNA or genomic DNA. Other suitable probes for use in the diagnostic assays of the invention are described herein.

An agent for detecting OAX protein is an antibody capable of binding to OAX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect OAX mRNA, protein, or genomic DNA in a biological sample in vitro as well as in vivo. For example, in vitro techniques for detection of OAX mRNA include Northern hybridizations and in situ hybridizations. In vitro techniques for detection of OAX protein include enzyme linked immunosorbent assays (ELISAs), Western blots, immunoprecipitations, and immunofluorescence. In vitro techniques for detection of OAX genomic DNA include Southern hybridizations. Furthermore, in vivo techniques for detection of OAX protein include introducing into a subject a labeled anti-OAX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting OAX protein, mRNA, or genomic DNA, such that the presence of OAX protein, mRNA or genomic DNA is detected in the biological sample, and comparing the presence of OAX protein, mRNA or genomic DNA in the control sample with the presence of OAX protein, mRNA or genomic DNA in the test sample.

The invention also encompasses kits for detecting the presence of OAX in a biological sample. For example, the kit can comprise: a labeled compound or agent capable of detecting OAX protein or mRNA in a biological sample; means for determining the amount of OAX in the sample; and means for comparing the amount of OAX in the sample with a standard. The compound or agent can be packaged in a suitable container. The kit can further comprise instructions for using the kit to detect OAX protein or nucleic acid.

Prognostic Assays

The diagnostic methods described herein can furthermore be utilized to identify subjects having or at risk of developing a disease or disorder associated with aberrant OAX expression or activity. For example, the assays described herein, such as the preceding diagnostic assays or the following assays, can be utilized to identify a subject having or at risk of developing a disorder associated with OAX protein, nucleic acid expression or activity. Alternatively, the prognostic assays can be utilized to identify a subject having or at risk for developing a disease or disorder. Thus, the invention provides a method for identifying a disease or disorder associated with aberrant OAX expression or activity in which a test sample is obtained from a subject and OAX protein or nucleic acid (e.g., mRNA, genomic DNA) is detected, wherein the presence of OAX protein or nucleic acid is diagnostic for a subject having or at risk of developing a disease or disorder associated with aberrant OAX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant OAX expression or activity. For example, such methods can be used to determine whether a subject can be effectively treated with an agent for a disorder. Thus, the invention provides methods for determining whether a subject can be effectively treated with an agent for a disorder associated with aberrant OAX expression or activity in which a test sample is obtained and OAX protein or nucleic acid is detected (e.g., wherein the presence of OAX protein or nucleic acid is diagnostic for a subject that can be administered the agent to treat a disorder associated with aberrant OAX expression or activity).

The methods of the invention can also be used to detect genetic lesions in an OAX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an OAX-protein, or the misexpression of the OAX gene. For example, such genetic lesions can be detected by ascertaining the existence of at least one of: (i) a deletion of one or more nucleotides from an OAX gene; (ii) an addition of one or more nucleotides to an OAX gene; (iii) a substitution of one or more nucleotides of an OAX gene, (iv) a chromosomal rearrangement of an OAX gene; (v) an alteration in the level of a messenger RNA transcript of an OAX gene, (vi) aberrant modification of an OAX gene, such as of the methylation pattern of the genomic DNA, (vii) the presence of a non-wild-type splicing pattern of a messenger RNA transcript of an OAX gene, (viii) a non-wild-type level of an OAX protein, (ix) allelic loss of an OAX gene, and (x) inappropriate post-translational modification of an OAX protein. As described herein, there are a large number of assay techniques known in the art which can be used for detecting lesions in an OAX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the OAX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an OAX gene under conditions such that hybridization and amplification of the OAX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an OAX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in OAX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in OAX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the OAX gene and detect mutations by comparing the sequence of the sample OAX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the OAX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type OAX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in OAX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an OAX sequence, e.g., a wild-type OAX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in OAX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control OAX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an OAX gene.

Furthermore, any cell type or tissue, preferably peripheral blood leukocytes, in which OAX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on OAX activity (e.g., OAX gene expression), as identified by a screening assay described herein can be administered to individuals to treat (prophylactically or therapeutically) disorders. The disorders include pathology such as inflammatory conditions associated with osteoarthritis and including inflammation of the joint and joint space, and degeneration of the cartilage matrix. In addition, OAX nucleic acids and their encoded polypeptides will be therapeutically useful for the prevention of pathologies associated with inflammation from osteoarthritis through gene therapy. Furthermore, OAX nucleic acids and their encoded polypeptides can be utilized to promote repair of the cartilage matrix (i.e., to promote the synthesis and deposition of oligomatrix support proteins) and for promoting chondrocyte growth and development and reduce cellular apoptosis of chondrocyte. Additional disorders wherein the associated expression of OAX in inflammatory conditions include those that mediate pro-inflammatory events (Crohns disease and ulcerative colitis), inflammatory lung disorders (asthma, chronic obstructive pulmonary disease (COPD), cystic fibrosis (CF), bronchiectasis and interstitial lung diseases) as well as Sjogren's syndrome, psoriasis, and rheumatoid arthritis.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of OAX protein, expression of OAX nucleic acid, or mutation content of OAX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of OAX protein, expression of OAX nucleic acid, or mutation content of OAX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an OAX modulator, such as a modulator identified by one of the exemplary screening assays described herein.

Monitoring of Effects During Clinical Trials

Monitoring the influence of agents (e.g., drugs, compounds) on the expression or activity of OAX (e.g., the ability to modulate aberrant cell proliferation and/or differentiation) can be applied not only in basic drug screening, but also in clinical trials. For example, the effectiveness of an agent determined by a screening assay as described herein to increase OAX gene expression, protein levels, or upregulate OAX activity, can be monitored in clinical trails of subjects exhibiting decreased OAX gene expression, protein levels, or downregulated OAX activity. Alternatively, the effectiveness of an agent determined by a screening assay to decrease OAX gene expression, protein levels, or downregulate OAX activity, can be monitored in clinical trails of subjects exhibiting increased OAX gene expression, protein levels, or upregulated OAX activity. In such clinical trials, the expression or activity of OAX and, preferably, other genes that have been implicated in, for example, a cellular proliferation or immune disorder can be used as a “read out” or markers of the immune responsiveness of a particular cell.

By way of example, and not of limitation, genes, including OAX, that are modulated in cells by treatment with an agent (e.g., compound, drug or small molecule) that modulates OAX activity (e.g., identified in a screening assay as described herein) can be identified. Thus, to study the effect of agents on cellular proliferation disorders, for example, in a clinical trial, cells can be isolated and RNA prepared and analyzed for the levels of expression of OAX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of OAX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an OAX protein, mRNA, or genomic DNA in the preadministration sample; (iii) obtaining one or more post-administration samples from the subject; (iv) detecting the level of expression or activity of the OAX protein, mRNA, or genomic DNA in the post-administration samples; (v) comparing the level of expression or activity of the OAX protein, mRNA, or genomic DNA in the pre-administration sample with the OAX protein, mRNA, or genomic DNA in the post administration sample or samples; and (vi) altering the administration of the agent to the subject accordingly. For example, increased administration of the agent may be desirable to increase the expression or activity of OAX to higher levels than detected, i.e., to increase the effectiveness of the agent. Alternatively, decreased administration of the agent may be desirable to decrease expression or activity of OAX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant OAX expression or activity. The disorders include include pathology such as inflammatory conditions associated with osteoarthritis, inflammation of the joint and joint space, and degeneration of the cartilage matrix. Other disorders wherein the associated expression of OAX in inflammatory conditions include those that mediate pro-inflammatory events (Crohns disease and ulcerative colitis), inflammatory lung disorders (asthma, chronic obstructive pulmonary disease (COPD), cystic fibrosis (CF), bronchiectasis and interstitial lung diseases) as well as Sjogren's syndrome, psoriasis, and rheumatoid arthritis and conditions of the like.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant OAX expression or activity, by administering to the subject an agent that modulates OAX expression or at least one OAX activity. Subjects at risk for a disease that is caused or contributed to by aberrant OAX expression or activity can be identified by, for example, any or a combination of diagnostic or prognostic assays as described herein. Administration of a prophylactic agent can occur prior to the manifestation of symptoms characteristic of the OAX aberrancy, such that a disease or disorder is prevented or, alternatively, delayed in its progression. Depending upon the type of OAX aberrancy, for example, an OAX agonist or OAX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating OAX expression or activity for therapeutic purposes. The modulatory method of the invention involves contacting a cell with an agent that modulates one or more of the activities of OAX protein activity associated with the cell. An agent that modulates OAX protein activity can be an agent as described herein, such as a nucleic acid or a protein, a naturally-occurring cognate ligand of an OAX protein, a peptide, an OAX peptidomimetic, or other small molecule. In one embodiment, the agent stimulates one or more OAX protein activity. Examples of such stimulatory agents include active OAX protein and a nucleic acid molecule encoding OAX that has been introduced into the cell. In another embodiment, the agent inhibits one or more OAX protein activity. Examples of such inhibitory agents include antisense OAX nucleic acid molecules and anti-OAX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an OAX protein or nucleic acid molecule. In one embodiment, the method involves administering an agent (e.g., an agent identified by a screening assay described herein), or combination of agents that modulates (e.g., up-regulates or down-regulates) OAX expression or activity. In another embodiment, the method involves administering an OAX protein or nucleic acid molecule as therapy to compensate for reduced or aberrant OAX expression or activity.

Stimulation of OAX activity is desirable in situations in which OAX is abnormally downregulated and/or in which increased OAX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The OAX nucleic acids and proteins of the invention are useful in potential prophylactic and therapeutic applications implicated in a variety of disorders including, but not limited to: disorders associated with OAX, with OAX, or with both OAX. As an example, a cDNA encoding the OAX protein of the invention may be useful in gene therapy, and the protein may be useful when administered to a subject in need thereof. By way of non-limiting example, the compositions of the invention will have efficacy for treatment of patients suffering from: In addition, OAX nucleic acids and their encoded polypeptides will be therapeutically useful for the prevention of pathologies associated with inflammation from osteoarthritis through gene therapy. Furthermore, OAX nucleic acids and their encoded polypeptides can be utilized to promote repair of the cartilage matrix (i.e., to promote the synthesis and deposition of oligomatrix support proteins) and for promoting chondrocyte growth and development and reduce cellular apoptosis of chondrocyte. Additional disorders wherein the associated expression of OAX in inflammatory conditions include those that mediate pro-inflammatory events (Crohns disease and ulcerative colitis), inflammatory lung disorders (asthma, chronic obstructive pulmonary disease (COPD), cystic fibrosis (CF), bronchiectasis and interstitial lung diseases) as well as Sjogren's syndrome, psoriasis, and rheumatoid arthritis.

Both the novel nucleic acid encoding the OAX protein, and the OAX protein of the invention, or nucleic acid or protein fragments, analogs, homologs or derivative thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

### EXAMPLES

The OAX nucleic acid and polypeptides described below as OA1-OA4 can be used with the methods of the present invention to ameliorate or treat the pathologies associated with osteoarthritis. By “treating” is meant the administration of a protein used in the present invention to a subject suffering from a pathology such as ostcoarthritis with the objective of providing a beneficial therapeutic effect. By “ameliorating” a pathology such as osteoarthritis, it is meant that a) in a subject in which the pathology is becoming more severe, one or more symptoms of the pathology cease becoming more severe and stabilize or improve; or b) in a subject in which the pathology is considered to be at a stable state, one or more symptoms of the pathology improve or become less severe. By “delaying the onset” of a pathology such as osteoarthritis, it is meant that administering a prophylactic dose or dosing regimen of a therapeutic agent such as the OAX and OAX proteins employed in the present invention results in the delay of appearance, or the delay of worsening, of one or more symptoms of a pathology such as osteoarthritis. Such a delay may be for an indeterminate period, in which the symptoms essentially never appear or never worsen, or it may be for a more limited period, in which the symptoms appear or worsen at a later time than would be expected, based on the experience of patients not treated by the compositions envisioned in the present methods, in the absence of administering the therapeutic agent.

Described below are the methods used to identify that OA1-OA4 are suitable as diagnostic markers, targets, and protein drugs for the treatment or diagnosis of osteoarthritis. Directly below, is a description of OA 1-OA4, and the role they each play in the treatment or diagnosis of osteoarthritis.

### Example 1

### Results from GeneCalling™ Experiments

Genes that are either upregulated or down regulated in diseased versus normal tissues have potential to be surrogate markers, drug targets, or to indicate pathways which might include targets. The proteins encoded by these genes may be suitable for use as diagnostic markers, targets, and protein drugs for osteoarthritis. Moreover, the difference in gene expression between ankles and knees will help explain the phenomenon that ankles seldomly develop OA, even in patients who have had hip and/or knee replacement therapy.

OA knee cartilage was collected from human subjects undergoing joint replacement therapy. Normal knee cartilage was collected from human donors post mortem. The OAX genes were identified following a TBLASTN (Altschul, S. F., Gish, W., Miller, W., Myers, E. W. & Lipman, D. J. (1990) J. Mol. Biol. 215, 403-410) search of Genbank human genomic DNA sequences. The individual sequences and results of the Genbank hits are described above. The osteoarthritis study established gene expression changes contributing to the development and progression of osteoarthritis utilizing the OAX genes identified. In addition, the study design identified the factors based on gene expression changes and known biological role, serve as protein therapeutics to slow the progression of osteoarthritis in human subjects.

GeneCalling® Technology: The GeneCalling® technology is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The three methods routinely used to confirm the identity of the gene fragment found to have altered expression in models of or patients with obesity and/or diabetes are described below.

A). Direct Sequencing

The differentially expressed gene fragment is isolated, cloned into a plasmid, and sequenced. Afterwards, the sequence information is used to design an oligonucleotide corresponding to either or both termini of the gene fragment. This oligonucleotide, when used in a competitive PCR reaction, will ablate the electropherographic band from which the sequence is derived.

B). Competitive PCR

In competitive PCR, the electropherographic peaks corresponding to the gene fragment of the gene of interest are ablated when a gene-specific primer (designed from the sequenced band or available databases) competes with primers in the linker-adaptors during the PCR amplification.

C). PCR with Perfect or Mismatched 3′ Nucleotides (TraPping)

This method utilizes a competitive PCR approach using a degenerate set of primers that extend one or two nucleotides into the gene-specific region of the fragment beyond the flanking restriction sites. As in the competitive PCR approach, primers that lead to the ablation of the electropherographic band add additional sequence information. In conjunction with the size of the gene fragment and the 12 nucleotides of sequence derived from the restriction sites, this additional sequence data can uniquely define the gene after database analysis.

### EQUIVALENTS

From the foregoing detailed description of the specific embodiments of the invention, it should be apparent that particular novel compositions and methods involving nucleic acids, polypeptides, antibodies, detection and treatment have been described. Although these particular embodiments have been disclosed herein in detail, this has been done by way of example for purposes of illustration only, and is not intended to be limiting with respect to the scope of the appended claims that follow. In particular, it is contemplated by the inventors that various substitutions, alterations, and modifications may be made as a matter of routine for a person of ordinary skill in the art to the invention without departing from the spirit and scope of the invention as defined by the claims. Indeed, various modifications of the invention in addition to those described herein will become apparent to those skilled in the art from the foregoing description and accompanying figures. Such modifications are intended to fall within the scope of the appended claims.

We claim:

1. A method of promoting the proliferation of chondrocyte cells comprising contacting the at least one chondrocyte cell with a composition comprising at least one polypeptide, wherein the polypeptide is an OAX polypeptide.

2. The method described in claim 1 wherein the cells are mammalian cells.

3. The method described in claim 1 wherein the cells are human cells.

4. A method of treating an inflammatory pathology in a subject comprising administering to the subject a composition comprising a polypeptide wherein the polypeptide is an OAX polypeptide.

5. The method described in claim 4 wherein the subject is a mammal.

6. The method described in claim 4 wherein the subject is a human.

7. The method described in claim 4 wherein the polypeptide comprises an OAX polypeptide, wherein the OAX polypeptide comprises
a) SEQ ID NO:2, 4, 6, or 8;
b) a variant of SEQ ID NO:2, 4, 6, or 8 wherein up to 15% of the residues provided in SEQ ID NO:2, 4, 6, or 8 are changed according to a conservative amino acid substitution;
c) a deletion mutant of SEQ ID NO:2, 4, 6, or 8; or
d) a variant of a deletion mutant of SEQ ID NO:2, 4, 6, or 8 wherein up to 15% of the residues provided in the deletion variant are changed according to a conservative amino acid substitution.

8. The method described in claim 4 wherein the inflammatory pathology is osteoarthritis.

9. The method described in claim 4 wherein the inflammatory pathology is an inflammatory condition occurring in the joint and joint space, and degeneration of the cartilage matrix and osteoarthritis.

10. The method described in claim 7 wherein the inflammatory pathology is an inflammatory condition is selected from the group consisting of inflammation of the joint and joint space, and degeneration of the cartilage matrix and osteoarthritis.

11. The method described in claim 7 wherein the polypeptide is administered to the subject intraperitoneally.

12. The method described in claim 7 wherein the polypeptide is administered to the subject intravenously.

13. The method described in claim 7 wherein the polypeptide comprises administered to the subject subcutaneously.

14. A method of promoting cartilage matrix repair in a subject comprising administering a OAX polypeptide.

15. The method described in claim 14 wherein the subject is a mammal.

16. The method described in claim 14 wherein the subject is a human.

17. A method of ameliorating a pro-inflammatory pathology in a subject comprising administering to the subject a composition said compositions comprising a combination of an OAX polypeptide, a growth factor, and a candidate therapeutic.

18. The method described in claim 17 wherein the subject is a mammal.

19. The method described in claim 17 wherein the subject is a human.

20. The method described in claim 17 wherein the composition is administered to the subject intravenously.

21. The method described in claim 17 wherein the composition is administered to the subject subcutaneously.

22. A method of preparing a pharmaceutical composition comprising combining at least one polypeptide effective in treating an inflammatory pathology with a pharmaceutically acceptable carrier, wherein the polypeptide is a OAX polypeptide.

23. The method described in claim 39 wherein the inflammatory pathology is osteoarthritis.
